(19) United States
(12) Patent Application Publication
(10) Pub. No.: US 2026/0345650 A1
(43) Pub. Date: Nov. 5, 2026

(54) OLIGOSACCHARIDE COMPOSITION FOR USE IN THE REDUCTION OF THE LEVEL OF AMMONIA

(71) Applicant: CLASADO LIMITED, Reading (GB)

(72) Inventors: Gediminas BALTULIONIS, Reading (GB); Kelly LITHERLAND, Reading (GB); Leunis Forrinus HARTHOORN, Reading (GB); Aleksandra Elzbieta MARUSZAK, Reading (GB)

(21) Appl. No.: 19/120,364

(22) PCT Filed: Oct. 12, 2023
(86) PCT No.: PCT/GB2023/052652

§ 371 Date: Apr. 11, 2025

(30) Foreign Application Priority Data
Oct. 12, 2022 (GB) 2215064.3

(51) Int. Cl. A61K 31/702; A61P 3/12

(52) U.S. Cl. CPC A61K 31/702; A61P 3/12

(57) ABSTRACT

A prebiotic oligosaccharide composition for use in reducing the level of ammonia. The composition comprises oligosaccharide compounds, for example galactooligosaccharide compounds, which includes: (a) at least 8 wt % Gal-(β1-3)-Gal-(β-4)-X^{a}; (b) at least 3 wt % Gal-(β1-3)-Gal-(β1-3)-X^{b}; and (c) at least 5 wt % Gal-(β1-3)-Gal-(β1-2)-X^{c}, based on the total weight of oligosaccharide compounds present in the composition; wherein X^{a}, X^{b} and X^{c} are each independently selected from monosaccharides. These compositions contain relatively high amounts of the oligosaccharide compounds (a), (b) and (c) and a relatively high amount of β1-3 Gal-Gal linkages, compared to known oligosaccharide compositions. The present invention also relates to a composition comprising oligosaccharide compounds for administration to the athletes and/or sports persons who are undergoing and/or are about to undergo physical training, and in particular those athletes and/or sports person on a high protein diet. A method of preparing said composition and the use of said composition as a dietary supplement or a medicament are also disclosed.

### FIELD

The present invention relates to galactooligosaccharides (GOS) compositions for use in reducing levels of ammonia in an individual in need thereof. The present invention also relates to methods of preparing said compositions and the formulation of said compositions as dietary supplements and/or medicaments. In particular the present invention relates to an oligosaccharide composition with a relatively high amount of certain beneficial oligosaccharides.

### BACKGROUND

Prebiotics are defined by the International Scientific Association for Probiotics and Prebiotics as “a substrate that is selectively utilized by host microorganisms conferring a health benefit”. Prebiotics are non-digestible dietary fibers that resist digestion and absorption until they reach the large intestine where they are fermented by members of the gut microbiome. They can modify the composition and function of the gut microbiome. A diet rich in dietary fiber/prebiotics has been shown to increase bacterial abundance and gut microbiome gene richness, as well as increasing the abundance of beneficial bacteria such as Bifidobacterium and Lactobacillus. Prebiotics also play multiple roles in suppressing gut pathogens, as an example, members of the Bifidobacterium and Lactobacillus genera produce lactic acid during prebiotic fermentation. Beneficial gut microbes utilize dietary fibers as an energy source. As a byproduct of this fermentation, they produce lactic acid and short-chain fatty acids (SCFAs), such as acetate, butyrate, and propionate. SCFAs are known to have several beneficial effects on human health including maintenance of the colonic epithelium; a potential role in regulating glucose homeostasis, lipid metabolism, and appetite regulation; and a role in regulating the immune system and inflammatory response.

Galactooligosaccharides (GOS) are an important and well-studied class of prebiotics. GOS has been shown to strongly stimulate the growth of bifidobacteria in the gut, as well as lactobacilli and Bacteroidetes, though to a lesser extent. Bifidobacteria are considered highly beneficial to the human host, primarily for their ability to produce SCFAs. Additionally, they are strongly associated with improvement in the intestinal epithelial barrier and intestinal permeability and play a beneficial role in host immunomodulation.

Known GOS prebiotic products contain a mixture of many oligosaccharide compounds in varying proportions, not all of which have the desired beneficial effects on the intestinal microbiota of the consumer. Although such products have shown some beneficial results in improving the health of patients, there remains a need for further improved oligosaccharide compositions to fully realise the potential benefits to consumers of such prebiotics and to provide a long-lasting and inexpensive treatments for a number of conditions and/or diseases.

### SUMMARY OF THE INVENTION

It is one aim of the present invention, amongst others, to provide a composition comprising oligosaccharide compounds that addresses at least one disadvantage of the prior art, whether identified here or elsewhere, or to provide an alternative to existing compositions. For instance it may be an aim of the present invention to provide a composition comprising oligosaccharide compounds which comprise a higher proportion of specific beneficial oligosaccharides than known oligosaccharide compositions.

According to aspects of the present invention, there is provided a composition method and use as set forth in the appended claims. Other features of the invention will be apparent from the dependent claims, and from the description which follows.

According to a first aspect of the present invention, there is provided a composition for reducing levels of ammonia, wherein the oligosaccharide compounds comprise:
(a) at least 8 wt % Gal-(β1-3)-Gal-(β1-4)-X^{a};
(b) at least 3 wt % Gal-(β1-3)-Gal-(β1-3)-X^{b}; and
(c) at least 5 wt % Gal-(β1-3)-Gal-(β1-2)-X^{c},
based on the total weight of oligosaccharide compounds present in the composition;
wherein X^{a}, X^{b} and X^{c} are each independently selected from monosaccharides.

In an embodiment related to the first aspect of the present invention, there is also provided a method of preventing, ameliorating or treating conditions and/or diseases associated with the elevated level of ammonia which comprises the administration of a composition comprising oligosaccharide compounds to a subject in need of such prevention, amelioration or treatment, wherein the oligosaccharide compounds comprise:
(a) at least 8 wt % Gal-(β1-3)-Gal-(β1-4)-X^{a};
(b) at least 3 wt % Gal-(β1-3)-Gal-(β1-3)-X^{b}; and
(c) at least 5 wt % Gal-(β1-3)-Gal-(β1-2)-X^{c},
based on the total weight of oligosaccharide compounds present in the composition;
wherein X^{a}, X^{b} and X^{c} are each independently selected from monosaccharides.

In a further embodiment related to the first aspect of the present invention, there is further provided a method of preventing, ameliorating or treating conditions and/or diseases related to elevated levels of ammonia in a subject in need of such prevention, amelioration or treatment, the method comprising:
i) determining whether the subject has elevated levels of levels of ammonia and/or whether the subject is suffering from a condition and/or disease associated with elevated levels of ammonia and
ii) administering a therapeutically effective amount of a composition comprising oligosaccharide compounds to the subject, wherein oligosaccharide compounds comprise:
   (a) at least 8 wt % Gal-(β1-3)-Gal-(β1-4)-X^{a};
   (b) at least 3 wt % Gal-(β1-3)-Gal-(β1-3)-X^{b}; and
   (c) at least 5 wt % Gal-(β1-3)-Gal-(β1-2)-X^{c},
   based on the total weight of oligosaccharide compounds present in the composition;
   wherein X^{a}, X^{b} and X^{c} are each independently selected from monosaccharides.

In a yet further embodiment of the present invention, there is provided the use of a composition for the manufacture of a medicament for the prevention, amelioration or treatment of a condition and/or disease associated with elevated levels of ammonia, wherein the composition comprises oligosaccharide compounds, said oligosaccharide compounds comprising:
(a) at least 8 wt % Gal-(β1-3)-Gal-(β1-4)-X^{a};
(b) at least 3 wt % Gal-(β1-3)-Gal-(β1-3)-X^{b}; and
(c) at least 5 wt % Gal-(β1-3)-Gal-(β1-2)-X^{c},
based on the total weight of oligosaccharide compounds present in the composition;
wherein X^{a}, X^{b} and X^{c} are each independently selected from monosaccharides.

In a further embodiment of the present invention, there is provided a composition for use in the prevention and/or treatment of fatigue in an athlete or sports person comprising oligosaccharide compounds, wherein the oligosaccharide compounds comprise:
(a) at least 8 wt % Gal-(β1-3)-Gal-(β1-4)-X^{a};
(b) at least 3 wt % Gal-(β1-3)-Gal-(β1-3)-X^{b}; and
(c) at least 5 wt % Gal-(β1-3)-Gal-(β1-2)-X^{c},
based on the total weight of oligosaccharide compounds present in the composition;
wherein X^{a}, X^{b} and X^{c} are each independently selected from monosaccharides.

A number of studies have highlighted the detrimental effects of elevated ammonia levels. Wilkinson, D. J. et. al. (2010) (Ammonia metabolism, the brain and fatigue: revisiting the link, Progress in Neurobiology, 91, (3), 200-219) discusses how hyperammonemia in exercise occurs as a result of an increased production by contracting muscle, through adenosine monophosphate (AMP) deamination (the purine nucleotide cycle) and branched chain amino acid (BCAA) deamination prior to oxidation. Plasma concentrations of ammonia during exercise often achieve or exceed those measured in liver disease patients, resulting in increased cerebral uptake which can result in disturbances in brain function, potentially through similar mechanisms underpinning pathology, which may impact on performance as fatigue or reduced function, especially during extreme exercise. Atbone, M. et. al. (2021) (The effect of acute moderate-intensity exercise on the serum and fecal metabolomes and the gut microbiota of cross-country endurance athletes, Nature Scientific Reports, 11, 3558) describes how exercise significantly increases fecal ammonia content, especially in individuals extreme exercise. Therefore, it follows that the composition of the present invention, would be suitable for use in the prevention and/or treatment of fatigue in an athlete or sports person, especially as it would target the gut and in turn reduce the amount of available ammonia which could be absorbed into the blood.

In a further embodiment, there is also provided a composition for use in the modulating the level of ammonia in an individual, wherein the composition comprises oligosaccharide compounds, and the oligosaccharide compounds comprise:
(a) at least 8 wt % Gal-(β1-3)-Gal-(β1-4)-X^{a};
(b) at least 3 wt % Gal-(β1-3)-Gal-(β1-3)-X^{b}; and
(c) at least 5 wt % Gal-(β1-3)-Gal-(β1-2)-X^{c},
based on the total weight of oligosaccharide compounds present in the composition;
wherein X^{a}, X^{b} and X^{c} are each independently selected from monosaccharides.

As used herein, the terms “treatment”, “treating”, “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting or slowing its development; and (c) relieving the disease and/or symptoms of the disease, i.e., causing regression of the disease.

The term “subject” used herein includes any human or nonhuman animal. The term “nonhuman animal” includes all mammals, such as nonhuman primates, sheep, dogs, cats, cows, horses.

A “therapeutically effective amount” refers to the amount of compounds, when administered to a subject for treating a disease, is sufficient to effect such treatment for the disease. The “therapeutically effective amount” will vary depending on the compound(s) used, the disease and its severity and the age, weight, etc., of the subject to be treated.

The term, “condition and/or disease associated with elevated levels of ammonia” refers to a range of conditions and/or diseases resulting from the elevated levels of ammonia in the body with identifiable symptoms. The elevated levels could be in the blood, gut, gut and/or intestinal lining.

Preferably the condition and/or disease associated with elevated levels of ammonia is hyperammonemia. The condition and/or disease associated with elevated levels of ammonia may be related to (or may be) any one of the following conditions and/or diseases: hepatic encephalopathy, Reye syndrome, urea cycle disorders, kidney failure, encephalopathy and liver failure.

The condition and/or disease associated with elevated levels of levels of ammonia may be in an individual who is an athlete or sports person who is undergoing (or about to undergo) physical exercise or training. The composition may be administered prior to, during, or after, undergoing exercise or physical activity. Alternatively, the composition may be administered according to a continuous dosage regime, such as once or twice daily. The therapeutically effective amount may be a standardised dose or a dose determined by individual factors such as age, body mass index (BMI) or the overall weight of the individual.

The term “performance” refers to the ability of an individual to perform certain physical tasks associated with sports and exercise, including, but not limited to reducing fatigue and improving endurance.

The term “athlete” or “sports person” refers to an individual actively engaged in sports or exercise, including individuals who are professionals or amateurs.

The present inventors have advantageously, and unexpectedly, found that supplementation using the composition of the invention reduced the levels of ammonia in trials. The composition acts systemically and studies have shown that the composition of the present invention can modulate levels of ammonia, which can result in a number of improvements such as the reduction of toxic by-products such (as p-cresol) due to proteolytic microbial activity in the gut. This is particularly relevant for individuals who are on a high protein diet or feeding regimen as they would have a higher proteolytic microbial activity due to the higher protein levels in the gut environment. The individuals may be on a high protein diet or feeding regimen for athletic purposes (e.g. to increase muscle mass and/or counteract muscle fatigue) or for medical purposes (e.g. to prevent or assist in the treatment of cachexia or sarcopenia). In certain embodiments, the composition may be for use in the prevention, amelioration or treatment of one or more of the following conditions: malnutrition, sarcopenia, cachexia and frailty.

These types of oligosaccharides having the specified linkages between the galactose units (denoted Gal) and terminal monosaccharides are believed to be particularly beneficial for the maintenance of a healthy human gut microbiota and therefore beneficial to the health of a subject. Known oligosaccharide compositions either do not contain each of these types of oligosaccharides or contain lower amounts of these oligosaccharides. Therefore the compositions of this first aspect provide advantages to the consumer in the establishment and maintenance of human gut microbiota when used as prebiotics.

The weight percentages of the specific oligosaccharide compounds discussed herein are based on the weight of all of the oligosaccharide compounds present in the whole composition. Therefore only the fraction of the composition which is provided by oligosaccharides, either components (a), (b), (c) or any other oligosaccharide present, is taken into account when determining the specified weight percentages. For this determination of oligosaccharide content, disaccharides are included, apart from lactose. The composition of this first aspect may contain other non-oligosaccharide components, including monosaccharides and lactose. These components are not taken into account when determining the specified weight percentages of the oligosaccharide compounds discussed herein. The specified amounts of oligosaccharide compounds in the composition can be referred to as a weight percentage of the oligosaccharide fraction of the composition of this first aspect.

Oligosaccharide compounds (a), (b) and (c) comprise X^{a}, X^{b} and X^{c} respectively, which are each independently selected from saccharides. The X^{a}, X^{b} and X^{c} groups may be considered to be saccharide units. Therefore the groups X^{a}, X^{b} and X^{c} can be considered terminal sugars of the oligosaccharide compounds. The groups X^{a}, X^{b} and X^{c} are suitably independently selected from monosaccharides. Any suitable monosaccharide unit which can form oligosaccharides with the galactose units of compounds (a), (b) and (c) can provide X^{a}, X^{b} and X^{c}. Suitably X^{a}, X^{b} and X^{c} are each independently selected from the following monosaccharides. Suitable monosaccharide units are selected from glucose (Glc), fucose (Fuc), arabinose (Ara), xylose (Xyl), rhamnose (Rha), mannose (Man), galactose (Gal), ribose (Rib), lyxose (Lyx), allose (All), altrose (Alt), gulose (Gul), idose (Ido), talose (Tal), psicose (Psi), fructose (Fru), sorbose (Sor), tagatose (Tag), galactosamine (GalN), glucosamine (GlcN) and N-Acetylglucosamine (GlcNAc) or a mixture thereof. Therefore each of compounds (a), (b) and (c) may comprise a mixture of oligosaccharide compounds having different respective X groups, for example either Glc or Fuc X groups.

In some embodiments, groups X^{a}, X^{b} and X^{c} are independently selected from the monosaccharides listed above, suitably independently selected from Glc, Fuc, Ara, Xyl, Rha and Man or a mixture thereof.

The saccharide units of the oligosaccharides in the composition of this first aspect may have either the D or the L enantiomeric form. Suitably the Gal saccharide units in components (a), (b) and (c) all have the D enantiomeric form. The components (a), (b) and (c) may therefore be as follows:
(a) D-Gal-(β1-3)-D-Gal-(β1-4)-X^{a};
(b) D-Gal-(β1-3)-D-Gal-(β1-3)-X^{b}; and
(c) D-Gal-(β1-3)-D-Gal-(β1-2)-X^{c}.

Suitably the Gal and the X^{a}, X^{b} and X^{c} saccharide units all have the D enantiomeric form. The components (a), (b) and (c) may therefore be as follows:
(a) D-Gal-(β1-3)-D-Gal-(β1-4)-D-X^{a};
(b) D-Gal-(β1-3)-D-Gal-(β1-3)-D-X^{b}; and
(c) D-Gal-(β1-3)-D-Gal-(β1-2)-D-X^{c}.

In some embodiments, each of X^{a}, X^{b} and X^{c} are Glc. Therefore compounds (a), (b) and (c) may be galactooligosaccharide compounds (GOS) and the composition of this first aspect may be referred to as a galactooligosaccharide composition. Such galactooligosaccharides may be formed by converting lactose to the stated oligosaccharides with a suitable galactosidase enzyme. In such embodiments the components (a), (b) and (c) are suitably as follows:
(a) Gal-(β1-3)-Gal-(β1-4)-Glc;
(b) Gal-(β1-3)-Gal-(β1-3)-Glc; and
(c) Gal-(β1-3)-Gal-(β1-2)-Glc.

Suitably the Gal and the Glc saccharide units all have the D enantiomeric form. The components (a), (b) and (c) may therefore be as follows:
(a) D-Gal-(β1-3)-D-Gal-(β1-4)-D-Glc;
(b) D-Gal-(β1-3)-D-Gal-(β1-3)-D-Glc; and
(c) D-Gal-(β1-3)-D-Gal-(β1-2)-D-Glc.

In some embodiments, each of X^{a}, X^{b} and X^{c} are a mixture of Glc and one or more of other saccharide units described above, for example Fuc, Ara, Xyl, Rha and Man. Therefore each of (a), (b) and (c) may comprise a mixture of oligosaccharide compounds having either Glc or one of the other saccharide units described above as the X group. In such embodiments, components (a), (b) and (c) may be formed by converting a mixture of lactose and an appropriate additional sugar, for example a monosaccharide selected from fucose, arabinose, xylose, rhamnose and mannose, to the oligosaccharides. Suitably each of X^{a}, X^{b} and X^{c} are a mixture of Glc and Fuc.

Other oligosaccharides in the composition comprising oligosaccharide compounds of this first aspect, besides components (a), (b) and (c) discussed above, may also comprise the saccharide unit “X” groups referred to above.

The composition of this first aspect comprises (a) at least 8 wt % Gal-(β1-3)-Gal-(β1-4)-X^{a}. Suitably the composition comprises at least 9 wt % of component (a) or at least 10 wt % of component (a).

Suitably the composition comprises up to 35 wt % of component (a), up to 30 wt % of component (a) or up to 25 wt % of component (a).

Suitably the composition comprises from 8 to 35 wt % of component (a), from 8 to 25 wt % of component (a) or from 10 to 20 wt % of component (a).

The composition of this first aspect comprises (b) at least 3 wt % Gal-(β1-3)-Gal-(β1-3)-X^{b}. Suitably the composition comprises at least 4 wt % of component (b) or at least 5 wt % of component (b).

Suitably the composition comprises up to 25 wt % of component (b), up to 20 wt % of component (b) or up to 15 wt % of component (b).

Suitably the composition comprises from 3 to 25 wt % of component (b), from 4 to 20 wt % of component (b) or from 4 to 10 wt % of component (b).

The composition of this first aspect comprises (c) at least 5 wt % Gal-(β1-3)-Gal-(β1-2)-X^{c}. Suitably the composition comprises at least 6 wt % of component (c).

Suitably the composition comprises up to 25 wt % of component (c), up to 20 wt % of component (c) or up to 15 wt % of component (c).

Suitably the composition comprises from 5 to 25 wt % of component (c), from 5 to 20 wt % of component (c) or from 6 to 10 wt % of component (c).

The above amounts are based on the total weight of oligosaccharide compounds present in the composition.

Suitably in the composition of this first aspect:
component (a) is present in an amount up to 35 wt %;
component (b) is present in an amount up to 25 wt %; and
component (c) is present in an amount up to 25 wt %;
based on the total weight of oligosaccharide compounds present in the composition.

Suitably in the composition of this first aspect:
component (a) is present in an amount from 8 to 25 wt %;
component (b) is present in an amount from 3 to 25 wt %; and
component (c) is present in an amount from 5 to 20 wt %;
based on the total weight of oligosaccharide compounds present in the composition.

In some embodiments, the composition of this first aspect comprises:
(a) from 8 to 25 wt % Gal-(β1-3)-Gal-(β1-4)-Glc;
(b) from 3 to 25 wt % Gal-(β1-3)-Gal-(β1-3)-Glc; and
(c) from 5 to 20 wt % Gal-(β1-3)-Gal-(β1-2)-Glc,
based on the total weight of oligosaccharide compounds present in the composition.

Suitably the ratio of the wt % of compound (a) to compound (b) is from 1:1 to 3:1, suitably from 1.5:1 to 2.5:1.

Suitably the ratio of the wt % of compound (a) to compound (c) is from 1:1 to 3:1, suitably from 1.5:1 to 2.5:1.

Suitably the ratio of the wt % of compound (b) to compound (c) is from 2:1 to 1:2, suitably from 1.5:1 to 1:1.5.

Suitably, in the composition of this first aspect, the oligosaccharide compounds comprise:
(d) at least 3 wt % Gal-(β1-3)-Gal-(β1-3)-Gal-(β1-4)-X^{d},

based on the total weight of oligosaccharide compounds present in the composition;
wherein X^{d}, is selected from saccharides.

X^{d} may be selected from the same monosaccharide units described above for X^{a}, X^{b} and X^{c}.

In some embodiments, X^{d} is Glc.

Suitably the Gal saccharide units of component (d) have the D enantiomeric form. Suitably the Gal and the X^{d} saccharide units all have the D enantiomeric form. The components (d) may therefore be as follows: D-Gal-(β1-3)-D-Gal-(β1-3)-D-Gal-(β1-4)-D-X^{d}.

In some embodiments, X^{d} is a mixture of Glc and one or more of other monosaccharide units, for example Fuc, Ara, Xyl, Rha and Man, suitably Fuc.

Suitably the composition comprises at least 4 wt % of component (d) or at least 5 wt % of component (d).

Suitably the composition comprises up to 25 wt % of component (d), up to 20 wt % of component (d) or up to 15 wt % of component (d).

Suitably the composition comprises from 3 to 25 wt % of component (d), from 4 to 20 wt % of component (d) or from 4 to 10 wt % of component (d).

Suitably in the composition of this first aspect:
component (a) is present in an amount from 8 to 25 wt %;
component (b) is present in an amount from 3 to 25 wt %;
component (c) is present in an amount from 5 to 20 wt %; and
component (d) is present in an amount from 3 to 25 wt %;
based on the total weight of oligosaccharide compounds present in the composition.

In some embodiments, the composition of this first aspect comprises:
(a) from 8 to 25 wt % Gal-(β1-3)-Gal-(β1-4)-Glc;
(b) from 3 to 25 wt % Gal-(β1-3)-Gal-(β1-3)-Glc;
(c) from 5 to 20 wt % Gal-(β1-3)-Gal-(β1-2)-Glc, and
(d) from 3 to 25 wt % Gal-(β1-3)-Gal-(β1-3)-Gal-(β1-4)-Glc,
based on the total weight of oligosaccharide compounds present in the composition.

Suitably, in the composition of this first aspect, the oligosaccharide compounds comprise:
(e) at least 5 wt % Gal-(β1-4)-Gal-(β1-4)-X^{e};
based on the total weight of oligosaccharide compounds present in the composition;
wherein X^{e} is selected from monosaccharides.

X^{e} may be selected from the same monosaccharide units described above for X^{a}, X^{b} and X^{c}.

Suitably the Gal saccharide units of component (e) have the D enantiomeric form. Suitably the Gal and the X^{e} saccharide units all have the D enantiomeric form. The components (e) may therefore be as follows: D-Gal-(β1-4)-D-Gal-(β1-4)-D-X^{e}.

In some embodiments, X^{e} is Glc.

In some embodiments, X^{e} is a mixture of Glc and one or more of other monosaccharide units, for example Fuc, Ara, Xyl, Rha and Man, suitably Fuc.

Suitably the composition comprises at least 6 wt % of component (e) or at least 7 wt % of component (e).

Suitably the composition comprises up to 25 wt % of component (e), up to 20 wt % of component (e) or up to 15 wt % of component (e).

Suitably the composition comprises from 5 to 25 wt % of component (e), from 5 to 20 wt % of component (e) or from 6 to 10 wt % of component (e).

Suitably in the composition of this first aspect:
component (a) is present in an amount from 8 to 25 wt %;
component (b) is present in an amount from 3 to 25 wt %;
component (c) is present in an amount from 5 to 20 wt %;
component (d) is present in an amount from 3 to 25 wt %; and
component (e) is present in an amount from 5 to 25 wt %;
based on the total weight of oligosaccharide compounds present in the composition.

In some embodiments, the composition of this first aspect comprises:
(a) from 8 to 25 wt % Gal-(β1-3)-Gal-(β1-4)-Glc;
(b) from 3 to 25 wt % Gal-(β1-3)-Gal-(β1-3)-Glc;
(c) from 5 to 20 wt % Gal-(β1-3)-Gal-(β1-2)-Glc, and
(d) from 3 to 25 wt % Gal-(β1-3)-Gal-(β1-3)-Gal-(β1-4)-Glc, and
(e) from 5 to 25 wt % Gal-(β1-4)-Gal-(β1-4)-Glc;
based on the total weight of oligosaccharide compounds present in the composition.

In the compositions of this first aspect, X^{a}, X^{b}, X^{c}, X^{d} and X^{e} are each independently selected from the monosaccharides described above.

In the compositions of this first aspect, X^{a}, X^{b}, X^{c}, X^{d} and X^{e} may each be independently selected from Glc, Fuc, Ara, Xyl, Rha and Man or a mixture thereof.

In some embodiments, X^{a}, X^{b}, X^{c}, X^{d} and X^{e} are each Glc.

In some embodiments, X^{a}, X^{b}, X^{c}, X^{d} and X^{e} each comprise Fuc. Suitably X^{a}, X^{b}, X^{c}, X^{d} and X^{e} are each a mixture of Glc and Fuc.

The oligosaccharide compounds of the compositions of this first aspect suitably have a relatively high proportion of β1-3 Gal-Gal linkages—mainly due to the presence of the components (a), (b), (c) and optionally (d) in the proportions discussed herein. The inventors have found that such relatively high proportions of β1-3 Gal-Gal linkages may be particularly advantageous for the uses of the composition discussed herein.

Suitably from 35 to 55% of the Gal-Gal linkages in the oligosaccharide compounds are 1-3 linkages, suitably from 40 to 55%, suitably from 40 to 50%.

Suitably from 40 to 60% of the Gal-Gal linkages in the oligosaccharide compounds are 1-4 linkages, suitably from 45 to 55%, suitably from 45 to 52%.

Suitably from 8 to 20% of the Gal-X linkages in the oligosaccharide compounds are 1-3 linkages, suitably from 10 to 18%, suitably from 12 to 16%, suitably wherein X is Glc.

Suitably from 15 to 25% of the Gal-X linkages in the oligosaccharide compounds are 1-4 linkages, suitably from 16 to 24%, suitably from 17 to 22%, suitably wherein X is Glc.

Suitably from 30 to 45% of the Gal-X linkages in the oligosaccharide compounds are 1-2 linkages, suitably from 32 to 43%, suitably from 34 to 41%, suitably wherein X is Glc.

Suitably from 20 to 36% of the Gal-X linkages in the oligosaccharide compounds are 1-6 linkages, suitably from 22 to 34%, suitably from 25 to 32%, suitably wherein X is Glc.

Suitably the Gal-X, linkages referred to above, for example for example Gal-Glu linkages, are p linkages, i.e β-glycosidic bonds.

Suitably the composition comprises at least 25 wt % of trisaccharides, based on the total weight of in the composition. Suitably the composition comprises at least 28 wt % of trisaccharides or at least 30 wt % of trisaccharides.

Suitably the composition comprises up to 70 wt % of trisaccharides, suitably up to 60 wt % trisaccharides or up to 50 wt % trisaccharides.

Suitably the composition comprises from 25 to 70 wt % of trisaccharides, suitably from 30 to 60 wt % trisaccharides or from 30 to 50 wt % trisaccharides.

Suitably the composition comprises at least 10 wt % of tetrasaccharides, based on the total weight of in the composition. Suitably the composition comprises at least 12 wt % of tetrasaccharides or at least 15 wt % of tetrasaccharides. Suitably the composition comprises at least 20 wt % tetrasaccharides or at least 25 wt % tetrasaccharides.

Suitably the composition comprises up to 40 wt % of tetrasaccharides, suitably up to 25 wt % tetrasaccharides or up to 20 wt % tetrasaccharides.

Suitably the composition comprises from 10 to 40 wt % of tetrasaccharides, suitably from 10 to 30 wt % tetrasaccharides or from 12 to 25 wt % tetrasaccharides.

Suitably the composition comprises from 30 to 50 wt % trisaccharides and from 10 to 25 wt % tetrasaccharides, based on the total weight of the composition.

As mentioned above, the determination of disaccharides present in the composition excludes lactose Therefore in some embodiments the composition suitably comprises up to 40 wt % of disaccharides, suitably up to 30 wt % disaccharides or up to 20 wt % disaccharides. Suitably the content of lactose in the composition of this first aspect is minimised. Suitably the composition is substantially free of lactose. Suitably the composition does not contain lactose.

Suitably the composition comprises from 10 to 40 wt % of disaccharides, suitably from 10 to 30 wt % disaccharides or from 10 to 20 wt % disaccharides.

Suitably the composition comprises from 10 to 40 wt % disaccharides, from 30 to 60 wt % trisaccharides and from 10 to 25 wt % tetrasaccharides, based on the total weight of the composition.

The following description relates to the content of disaccharides in the oligosaccharide compounds.

Suitably the oligosaccharide compounds comprise up to 40 wt % of disaccharides, suitably up to 30 wt % disaccharides or up to 20 wt % disaccharides.

Suitably the oligosaccharide compounds comprise from 0 to 40 wt % of disaccharides, suitably from 10 to 30 wt % disaccharides or from 10 to 20 wt % disaccharides.

Suitably the oligosaccharide compounds comprise at least 25 wt % of trisaccharides, based on the total weight of oligosaccharide compounds present in the composition. Suitably the oligosaccharide compounds comprise at least 30 wt % of trisaccharides or at least 33 wt % of trisaccharides.

Suitably the oligosaccharide compounds comprise up to 75 wt % of trisaccharides, suitably up to 65 wt % trisaccharides or up to 55 wt % trisaccharides.

Suitably the oligosaccharide compounds comprise from 25 to 75 wt % of trisaccharides, suitably from 30 to 65 wt % trisaccharides or from 34 to 55 wt % trisaccharides.

Suitably the oligosaccharide compounds comprise at least 10 wt % of tetrasaccharides, based on the total weight of oligosaccharide compounds present in the composition. Suitably the oligosaccharide compounds comprise at least 12 wt % of tetrasaccharides or at least 15 wt % of tetrasaccharides.

Suitably the oligosaccharide compounds comprise up to 45 wt % of tetrasaccharides, suitably up to 35 wt % tetrasaccharides or up to 30 wt % tetrasaccharides.

Suitably the oligosaccharide compounds comprise from 10 to 45 wt % of tetrasaccharides, suitably from 10 to 35 wt % tetrasaccharides or from 15 to 30 wt % tetrasaccharides.

Suitably the oligosaccharide compounds comprise from 0 to 40 wt % disaccharides and from 30 to 75 wt % trisaccharides, based on the total weight of oligosaccharide compounds present in the composition.

Suitably the oligosaccharide compounds comprise from 0 to 40 wt % disaccharides, from 30 to 75 wt % trisaccharides and from 10 to 45 wt % tetrasaccharides, based on the total weight of oligosaccharide compounds present in the composition.

The composition of this first aspect suitably comprises at least 50 wt % of oligosaccharide compounds, including components (a), (b), (c) and optionally (d) and (e). Suitably the composition comprises at least 55 wt % of oligosaccharide compounds, suitably at least 60 wt %, based on the total weight of the composition.

Suitably the composition comprises up to 100 wt % oligosaccharide compounds, suitably up to 95 wt %, up to 90 wt % or up to 85 wt % oligosaccharide compounds.

Suitably the composition comprises from 50 to 100 wt % oligosaccharide compounds, suitably from 55 to 95 wt %, or from 60 to 85 wt % oligosaccharide compounds.

In some embodiments, the composition is in the form of a syrup. The syrup suitably comprises at least 50 wt % of oligosaccharide compounds, at least 55 wt % of oligosaccharide compounds, or at least 60 wt %. Suitably the syrup comprises from 50 to 75 wt % of oligosaccharide compounds, suitably from 55 to 70 wt % or from 60 to 70 wt % oligosaccharide compounds.

The syrup may comprise a significant amount of monosaccharides, for example glucose and/or galactose. The syrup may comprise from 15 to 30 wt % monosaccharides, suitably from 20 to 28 wt % monosaccharides or from 21 to 25 wt % monosaccharides, for example glucose and/or galactose.

The syrup suitably comprises from 20 to 30 wt % water, suitably from 22 to 28 wt % water.

The syrup may also contain lactose, for example 4 to 14 wt % lactose.

In some embodiments, the composition is in the form of a powder. The powder suitably comprises at least 60 wt % of oligosaccharide compounds, suitably at least 70 wt % or at least 75 wt % of oligosaccharide compounds. Suitably the powder comprises from 60 to 100 wt % of oligosaccharide compounds, suitably from 70 to 95 wt % or from 75 to 90 wt % oligosaccharide compounds.

The powder suitably comprises a reduced amount of monosaccharides, for example glucose and/or galactose, compared to the syrup discussed above. The powder may comprise from 1 to 10 wt % monosaccharides, suitably from 2 to 8 wt % monosaccharides or from 3 to 7 wt % monosaccharides, suitably approximately 5 wt %, for example of glucose and/or galactose.

The powder suitably comprises from 1 to 10 wt % water, suitably from 3 to 6 wt % water.

The composition of this first aspect may have been purified to remove monosaccharides and optionally disaccharides from the composition.

The composition of this first aspect may have been fractionated to separate the oligosaccharide components of the composition according to their molecular weight, for example to remove disaccharides from the composition or to isolate trisaccharides from the other oligosaccharide components. This may be carried out by any suitable method known in the art, for example high-performance liquid chromatography. The composition produced by such a fractionation may be referred to as an oligosaccharide fraction or a GOS fraction.

In such embodiments, the composition (or oligosaccharide fraction) suitably comprises at least 70 wt % of trisaccharides, tetrasaccharides and higher oligosaccharides, suitably at least 80 wt % or at least 90 wt %. Such higher oligosaccharides have a degree of polymerisation of 5 and above.

Suitably the composition comprises at least 95 wt % of trisaccharides, tetrasaccharides and higher oligosaccharides. Suitably the composition consists or consists essentially of trisaccharides, tetrasaccharides and higher oligosaccharides.

In such embodiments, the composition suitably comprises from 40 to 70 wt % of trisaccharides, suitably from 45 to 70 wt % trisaccharides or from 50 to 70 wt % trisaccharides.

Suitably the oligosaccharide compounds comprise from 15 to 50 wt % of tetrasaccharides, suitably from 15 to 40 wt % tetrasaccharides or from 20 to 40 wt % tetrasaccharides.

Suitably the oligosaccharide compounds comprise from 5 to 25 wt % of higher oligosaccharides, suitably from 5 to 20 wt % higher oligosaccharides or from 10 to 20 wt % higher oligosaccharides.

Suitably the composition comprises from 40 to 70 wt % trisaccharides, from 15 to 40 wt % tetrasaccharides and 5 to 25 wt % of higher oligosaccharides, based on the total weight of the composition. Suitably the composition comprises from 50 to 70 wt % trisaccharides, from 20 to 40 wt % tetrasaccharides and from 10 to 20 wt % higher oligosaccharides, based on the total weight of the composition.

In such embodiments the composition (or oligosaccharide fraction) suitably comprises the components (a), (b) and (c) as described above in the following amounts:
(a) at least 8 wt % Gal-(β1-3)-Gal-(β1-4)-X^{a};
(b) at least 3 wt % Gal-(β1-3)-Gal-(β1-3)-X^{b}; and
(c) at least 5 wt % Gal-(β1-3)-Gal-(β1-2)-X^{c},

based on the total weight of oligosaccharide compounds present in the composition;
wherein X^{a}, X^{b} and X^{c} are each independently selected from monosaccharides.

Suitably the composition (or oligosaccharide fraction) comprises the components (a), (b) and (c) as described above in the following amounts:
(a) at least 10 wt % Gal-(β1-3)-Gal-(β1-4)-X^{a};
(b) at least 5 wt % Gal-(β1-3)-Gal-(β1-3)-X^{b}; and
(c) at least 7 wt % Gal-(β1-3)-Gal-(β1-2)-X^{c}.

The composition may comprise components (d) and/or component (e) as described above.

The composition suitably comprises said components in the ratios discussed above.

In some embodiments, the composition of this first aspect is a trisaccharide and tetrasaccharide fractionated product (which may be referred to as a DP3/DP4 fraction).

In such embodiments, the composition (or DP3/DP4 fraction) suitably comprises at least 70 wt % of trisaccharides and tetrasaccharides, suitably at least 80 wt % or at least 90 wt %. Suitably the composition consists or consists essentially of trisaccharides and tetrasaccharides.

In such embodiments, the composition suitably comprises from 50 to 80 wt % of trisaccharides, suitably from 55 to 75 wt % trisaccharides or from 60 to 75 wt % trisaccharides.

Suitably the oligosaccharide compounds comprise from 20 to 50 wt % of tetrasaccharides, suitably from 25 to 45 wt % tetrasaccharides or from 25 to 40 wt % tetrasaccharides.

Suitably the composition comprises from 50 to 80 wt % trisaccharides and from 20 to 50 wt % tetrasaccharides, based on the total weight of the composition. Suitably the composition comprises from 60 to 75 wt % trisaccharides and from 25 to 40 wt % tetrasaccharides, based on the total weight of the composition.

In such embodiments the composition (or oligosaccharide fraction) suitably comprises the components (a), (b) and (c) as described above in the following amounts:
(a) at least 8 wt % Gal-(β1-3)-Gal-(β1-4)-X^{a};
(b) at least 3 wt % Gal-(β1-3)-Gal-(β1-3)-X^{b}; and
(c) at least 5 wt % Gal-(β1-3)-Gal-(β1-2)-X^{c},
based on the total weight of oligosaccharide compounds present in the composition;
wherein X^{a}, X^{b} and X^{c} are each independently selected from monosaccharides.

Suitably the composition (or oligosaccharide fraction) comprises the components (a), (b) and (c) as described above in the following amounts:
(a) at least 10 wt % Gal-(β1-3)-Gal-(β1-4)-X^{a};
(b) at least 5 wt % Gal-(β1-3)-Gal-(β1-3)-X^{b}; and
(c) at least 7 wt % Gal-(β1-3)-Gal-(β1-2)-X^{c}.

The composition may comprise components (d) and/or component (e) as described above.

The composition suitably comprises said components in the ratios discussed above.

In some embodiments, the composition of this first aspect is a trisaccharide fractionated product (which may be referred to as a DP3 fraction). Such a trisaccharide fractionated product may be obtained by the known fractionation methods referred to above. Such a composition suitably comprises at least 70 wt % of trisaccharides, suitably at least 80 wt % or at least 90 wt %. Suitably the composition comprises at least 95 wt % of trisaccharides.

In such embodiments the composition (or oligosaccharide fraction) suitably comprises the components (a), (b) and (c) as described above in the following amounts:
(a) at least 8 wt % Gal-(β1-3)-Gal-(β1-4)-X^{a};
(b) at least 3 wt % Gal-(β1-3)-Gal-(β1-3)-X^{b}; and
(c) at least 5 wt % Gal-(β1-3)-Gal-(β1-2)-X^{c},
based on the total weight of oligosaccharide compounds present in the composition;
wherein X^{a}, X^{b} and X^{c} are each independently selected from saccharides.

Suitably the composition (or oligosaccharide fraction) comprises the components (a), (b) and (c) as described above in the following amounts:
(a) at least 10 wt % Gal-(β1-3)-Gal-(β1-4)-X^{a};
(b) at least 5 wt % Gal-(β1-3)-Gal-(β1-3)-X^{b}; and
(c) at least 7 wt % Gal-(β1-3)-Gal-(β1-2)-X^{c}.

The composition may comprise component (e) as described above.

The composition suitably comprises said components in the ratios discussed above.

The composition of this first aspect may be for use as, and incorporated into, a food supplement product for ingestion by a consumer Such a product may be selected from the group consisting of dairy products (for example, liquid milk, dried milk powder such as whole milk powder, skimmed milk powder, fat filled milk powders, whey powders, infant formula, ice cream, yoghurt, cheese, fermented dairy products), beverages, sport drinks, infant foods, cereals, bread, biscuits, confectionary, cakes, food supplements, dietary supplements, medical food/nutrition, food for specific medical purposes, animal feeds, poultry feeds or indeed any other food or beverage.

The composition of this first aspect may be incorporated into a synbiotic composition. Such a synbiotic composition is suitably a mixture comprising live microorganisms and substrate(s) selectively utilized by host microorganisms that confers a health benefit on the host, i.e. a probiotic and a prebiotic.

The composition of this first aspect may be, for use as, and in the form of, a pharmaceutical or nutraceutical composition comprising at least one carrier, excipient, or diluent.

Suitable further components of pharmaceutical or nutraceutical compositions and methods of preparing such pharmaceutical or nutraceutical compositions are known in the art.

The composition may be administered in a single dose or in multiple doses. A suitable frequency of administration may be at least once per day, every other day, once per week, once every two, three, or four weeks, once every month, two months, or once every three to six months. The composition may be administered over a period of at least a week, at least a month, at least three to six months, at least one, two, three, four, or five years, or over the course of the disease, or the lifetime of the subject.

It will be apparent to the skilled addressee that the administration of the composition will be optimised during clinical trials.

Compositions of the invention can be formulated into pharmaceutical compositions by combination with appropriate pharmaceutically acceptable carriers, pharmaceutically acceptable diluents, or pharmaceutically acceptable excipients, and can be formulated into preparations in solid, semi-solid, or liquid forms, such as tablets, capsules, powders, granules and solutions.

Pharmaceutically acceptable carriers, excipients, or diluents may include, for example: water, saline, dextrose, maltodextrin, glycerol, ethanol, a salt, e.g., NaCl, MgCl₂, KCl, MgSO₄, etc.; a buffering agent, e.g., a phosphate buffer, a citrate buffer, a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; glycerol; and the like.

Pharmaceutically acceptable carriers, excipients and diluents are nontoxic to recipients at the dosages and concentrations employed, and can for example include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and valine, and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

For oral preparations, the composition of the invention may include appropriate additives to make tablets, powders, granules or capsules, for example, with 30 conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The pharmaceutical composition can be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization).

A tonicity agent can be included in the formulation to modulate the tonicity of the formulation. Exemplary tonicity agents include sodium chloride, potassium chloride, glycerin and any component from the group of amino acids, sugars as well as combinations thereof. In some embodiments, the aqueous formulation is isotonic, although hypertonic or hypotonic solutions can be suitable. The term “isotonic” denotes a solution having the same tonicity as some other solution with which it is compared, such as a physiological salt solution or serum.

The composition may modulate the abundance of a bacterial genus present in the gut. In some embodiments, the composition modulates the abundance of a bacterial genus present in one or both of the small intestine or large intestine. In some embodiments, the composition modulates the abundance of a bacterial genus predominant in the small intestine selected from the group of genus Achromobacter, Agrobacterium, Blautia, Burkholderia, Coprococcus, Cryocola, Enterococcus, Eubacterium, Holdemania, Lactococcus, Mycobacterium, Pseudoramibacter, Ralstonia, Sphingomonas, Streptococcus, and Turicibacter.

According to a second aspect of the present invention, there is provided a method of preparing a composition comprising oligosaccharide compounds according to the first aspect, the method comprising the steps of:
(i) providing a source of saccharide compounds;
(ii) treating the source of saccharide compounds with one or more galactosidase enzyme to at least partially convert the source of saccharide compounds to the oligosaccharide compounds.

Suitably the steps of the method of this second aspect are carried out in the order step (i) followed by step (ii).

Suitably the source of saccharide compounds comprises lactose, lactulose or epilactose. Suitably the source of saccharide compounds comprises lactose. The source of saccharide may be lactose, for example a lactose syrup which may be derived from cow's milk. The lactose may be heat treated.

In some embodiments, no further saccharides, such as monosaccharides or disaccharides are added to the source of saccharide. In such embodiments, the method produces galactooligosaccharide compounds.

In some embodiments, the source of saccharide compounds comprises at least one additional saccharide. Suitably the at least one additional saccharide provides the oligosaccharide compounds with an alternative terminal monosaccharide unit, as discussed above. Suitably the at least one additional saccharide is a source of such a monosaccharide unit. The at least one additional saccharide which is a source of such a monosaccharide unit may be a monosaccharide or may be a higher sugar, such as a disaccharide. The at least one additional saccharide may be a source of a monosaccharide selected from glucose (Glc), fucose (Fuc), arabinose (Ara), xylose (Xyl), rhamnose (Rha), mannose (Man), galactose (Gal), ribose (Rib), lyxose (Lyx), allose (All), altrose (Alt), gulose (Gul), idose (Ido), talose (Tal), psicose (Psi), fructose (Fru), sorbose (Sor), tagatose (Tag), galactosamine (GalN), glucosamine (GlcN) and N-Acetylglucosamine (GlcNAc) or a mixture thereof.

The at least one additional saccharide may be one or more of the monosaccharides listed above.

In such embodiments, the method produces oligosaccharides having one or more of the above monosaccharides as the terminal sugar unit.

In some embodiments, the at least one additional saccharide may be selected from Fucose, Arabinose, Xylose, Rhamnose, Mannose or mixtures thereof. The source of saccharides may comprise lactose and one or more sources of said monosaccharides. In such embodiments, the method produces oligosaccharides having as a terminal sugar unit selected from Glc, Fuc, Ara, Xyl, Rha and Man, or mixtures thereof.

Step (ii) of the method involves treating the source of saccharide compounds with at least one galactosidase enzyme. The galactosidase enzyme may be an alpha- or beta-galactosidase enzyme, depending on whether alpha or beta linkages between the saccharide units of the oligosaccharide compounds is required. Suitably the enzyme exhibits galactosyltransferase (transgalactosidic) activity and forms alpha- or beta-linkages between sugar units in the source of saccharide compounds. This results in the synthesis of oligosaccharide compounds with two or more galactose units derived from lactose. Suitably step (ii) is carried out until conversion of the source of saccharides to the oligosaccharide compounds is complete.

Step (ii) may involve treating the source of saccharide compounds with one or more additional enzymes which are not galactosidase enzymes.

Suitably the method comprises a step (iii) of separating the galactosidase enzyme from the composition comprising oligosaccharide compounds. Step (iii) may involve removing the enzyme by filtration, for example by nanofiltration.

The composition comprising oligosaccharide compounds produced in step (iii) may be heat treated.

In some embodiments, the composition is evaporated to reduce the water content to provide the final composition comprising oligosaccharide compounds as a syrup, as discussed above in relation to the first aspect.

In some embodiments, glucose is removed from the composition produced by step (iii) before evaporation. This suitably lowers the glucose content of the composition from 20-30 wt % to below 10 wt %, suitably approximately 5 wt % or lower. The water content of the composition is then reduced by evaporation and the product dried to provide the final composition comprising oligosaccharide compounds as a powder, as discussed above in relation to the first aspect.

It will also be appreciated that a person skilled in the relevant art could produce a composition comprising oligosaccharide compounds according to the first aspect, i.e. containing the specified amounts of particular oligosaccharide compounds, by combining said oligosaccharides obtained and isolated from different sources in the required amounts.

The invention is described below, by way of example only, with reference to the accompanying figures in which:

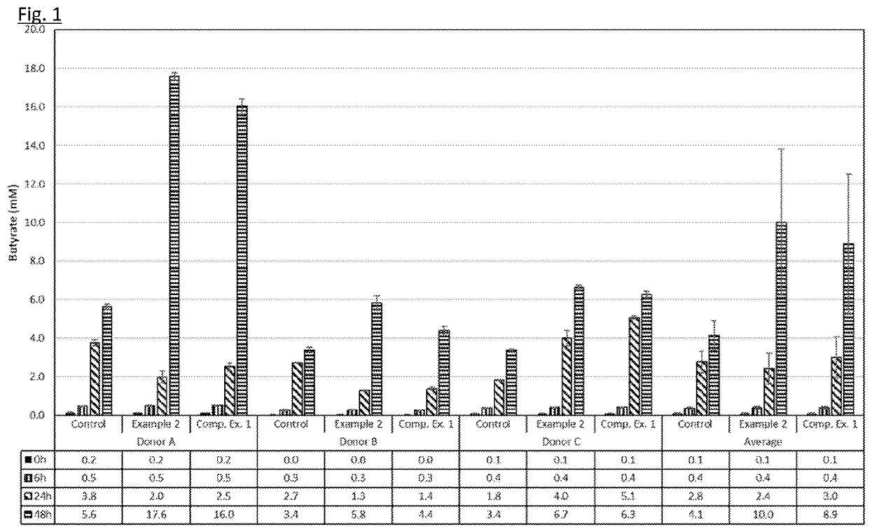

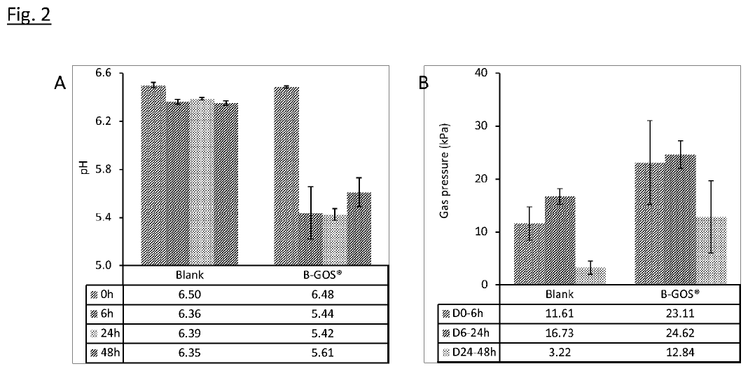

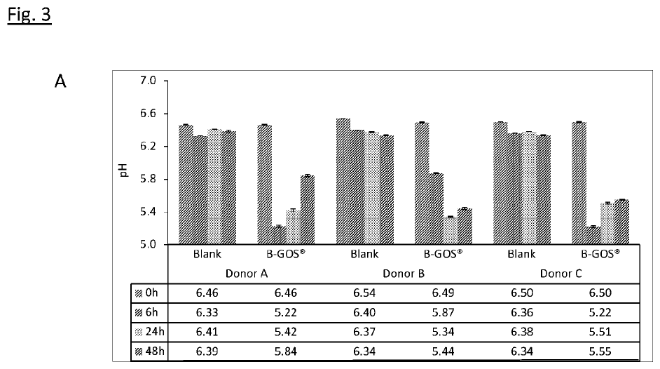

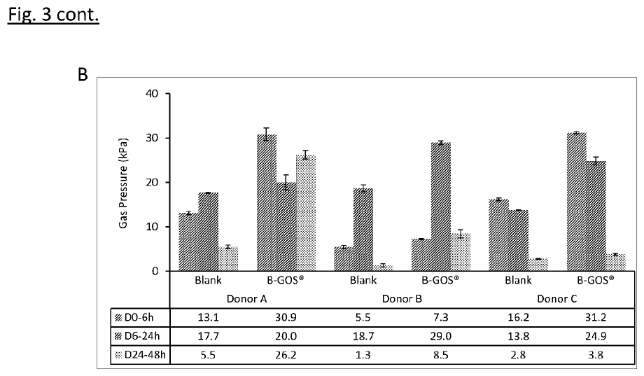

FIG. 1 is a graph illustrating the results of the butyrate analysis across three donors after being administered the oligosaccharide of the present invention alongside a comparative oligosaccharide in Comparative Example 1.

FIG. 2 are graphs showing the overall microbial community activity (acidification and gas production) shown as (A) pH and (B) gas pressure. Measurements were collected in triplicate in Example 4. Data for average values were derived using data from Donors A/B/C. Error bars represent standard deviation. GOS=Bimuno® galactooligosaccharides.

FIG. 3 are graphs showing the individual microbial community activity (acidification and gas production) shown as (A) pH and (B) gas pressure across all donors in Example 4.

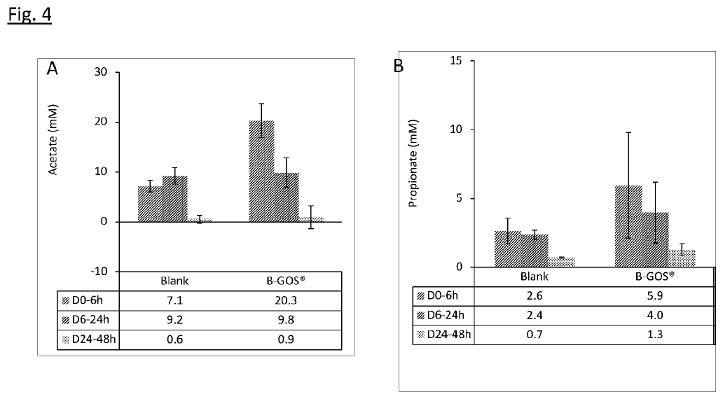

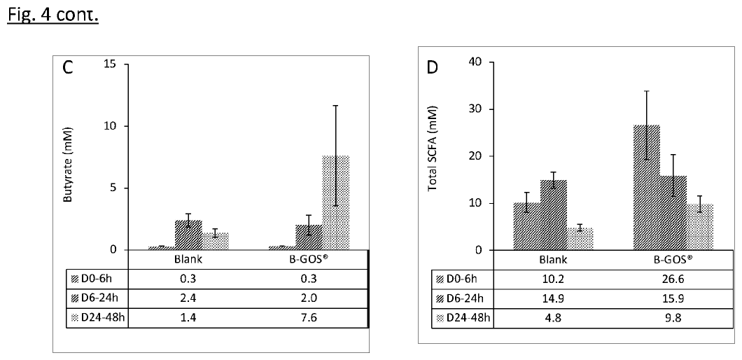

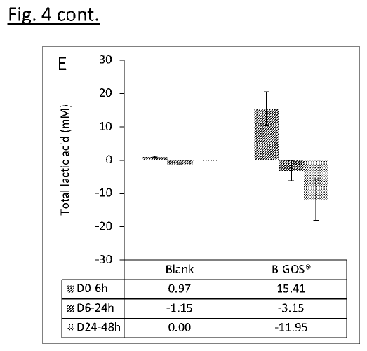

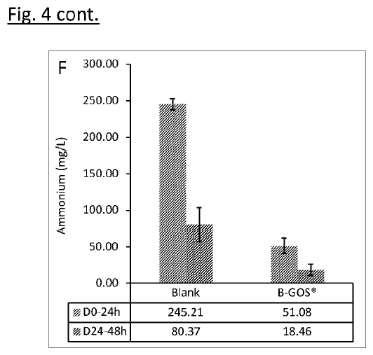

FIG. 4 are graphs showing microbial metabolic activity (A) acetate, (B) propionate, (C) butyrate, (D) total SCFA, (E) lactate and (F) ammonium. Measurements were collected in triplicate in Example 4. Data for average values were derived using data from Donors A/B/C. Error bars represent standard deviation. SCFA=short-chain fatty acid.

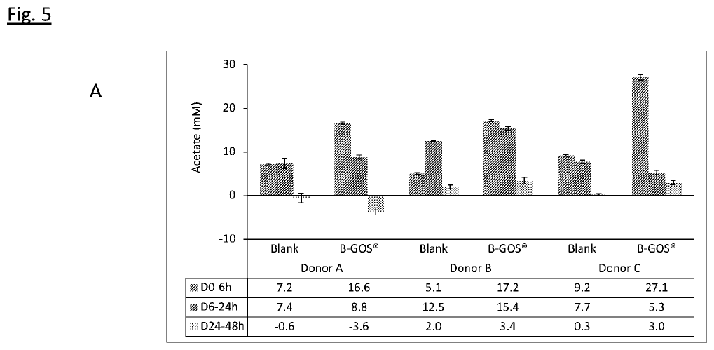

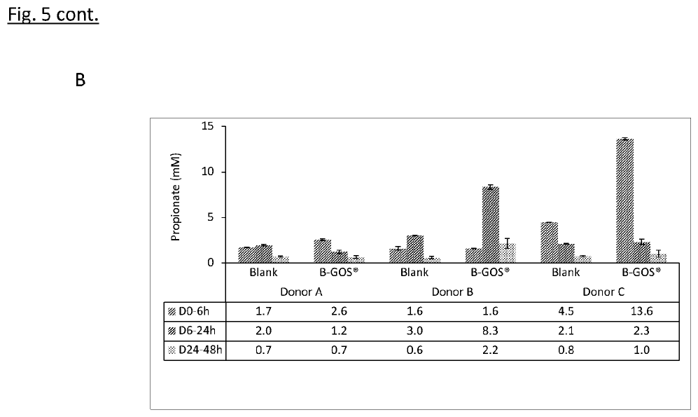

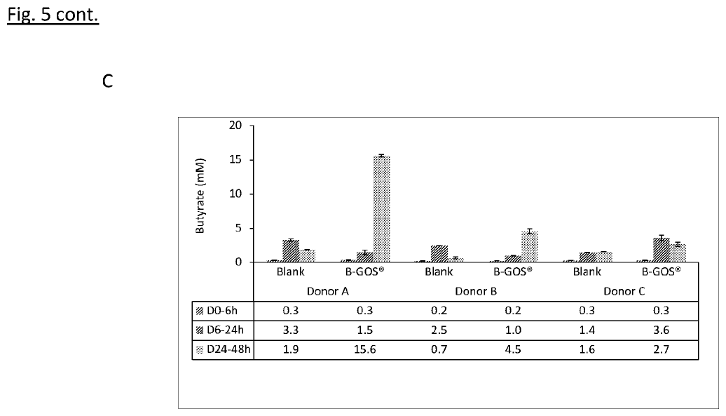

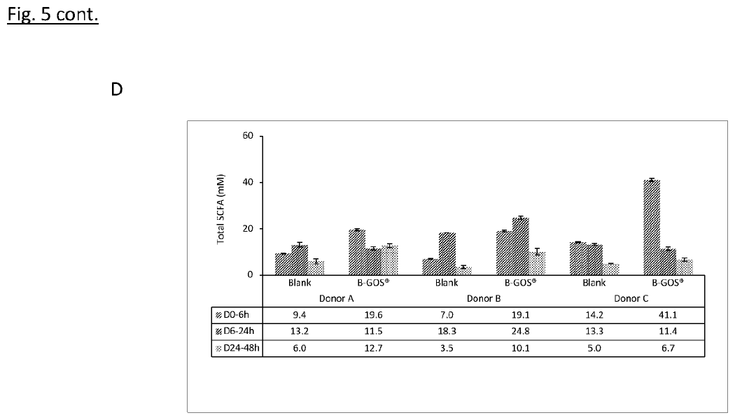

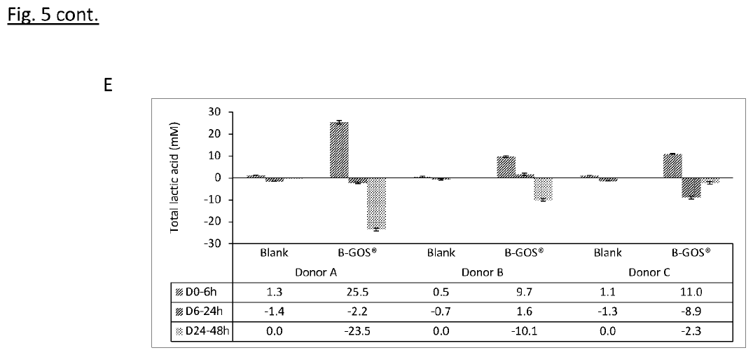

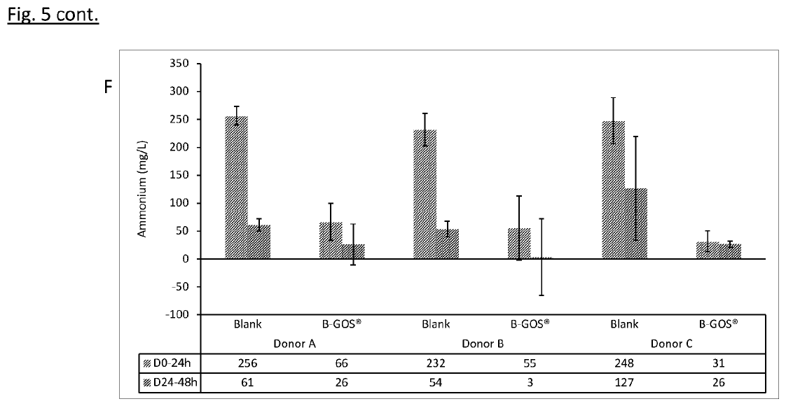

FIG. 5 are graphs showing microbial metabolic activity (A) acetate, (B) propionate, (C) butyrate, (D) total SCFA, (E) lactate and (F) ammonium for individual donors in Example 4. Measurements were collected in triplicate. Error bars represent standard deviation. SCFA=short-chain fatty acid.

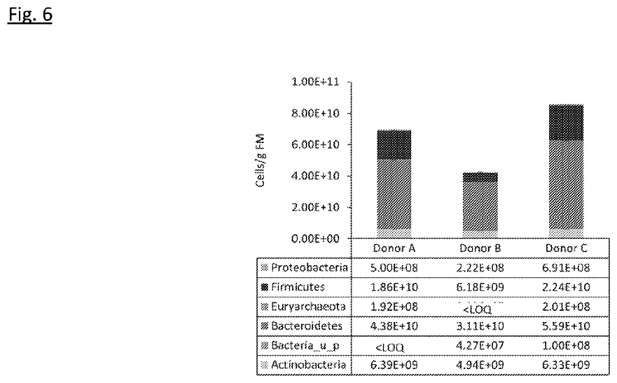

FIG. 6 is a graph showing the representation of major phyla in the fecal microbiota of each donor at 0 h in Example 4. Samples were assayed in triplicate. FM=fecal matter; LOQ=limit of quantification

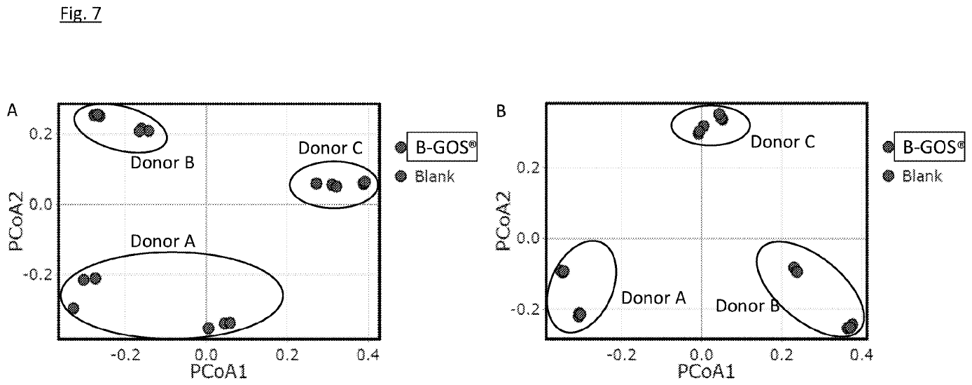

FIG. 7 are plots showing the Principal Coordinate Analysis of species data using Bray-Curtis distance for each donor at (A) 6 h after treatment (relative data) and (B) 24 h after treatment (absolute data) in Example 4. Each dot represents one replicate.

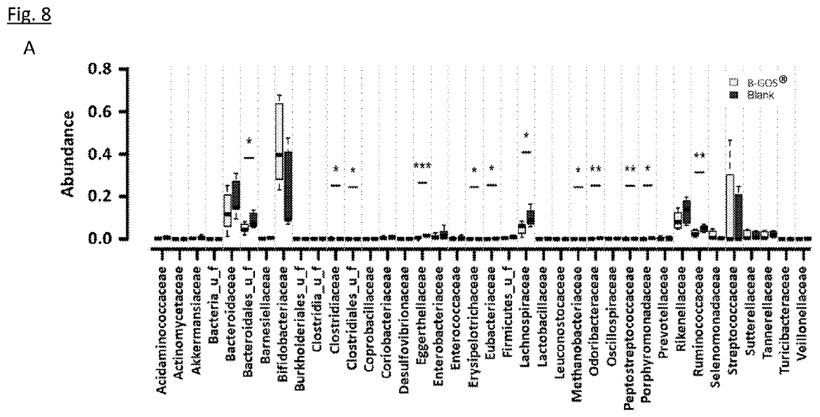

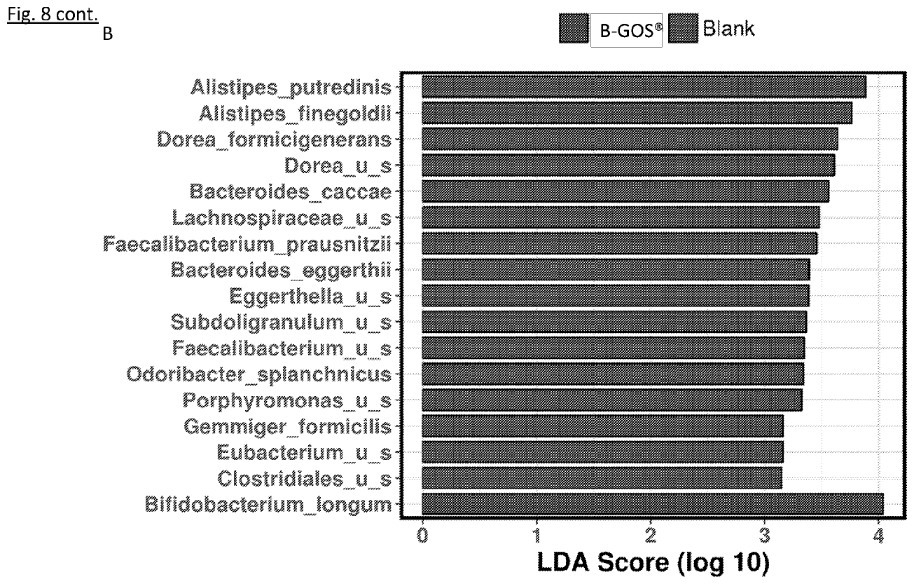

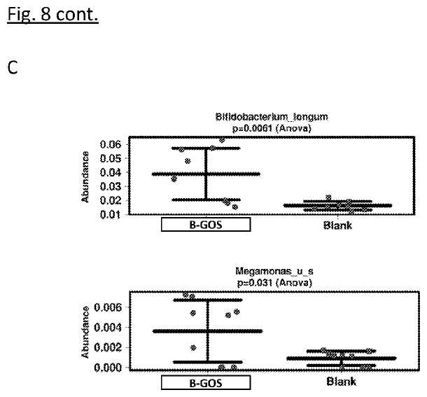

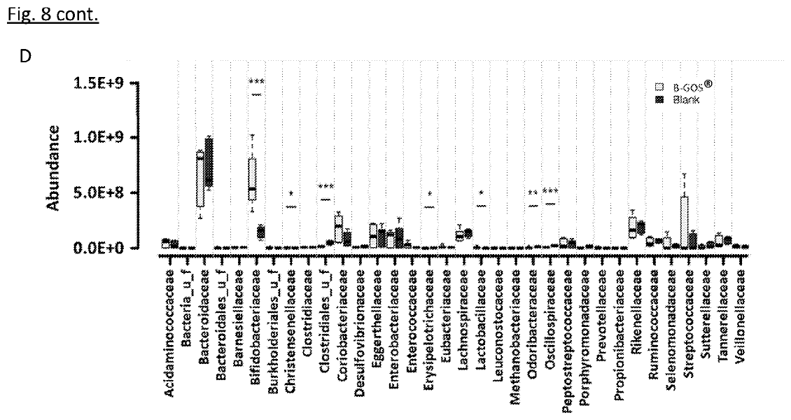

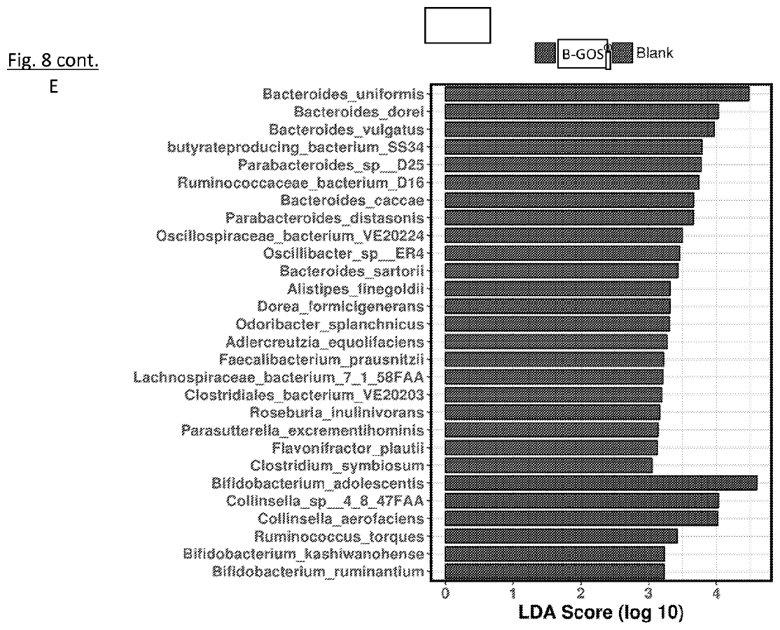

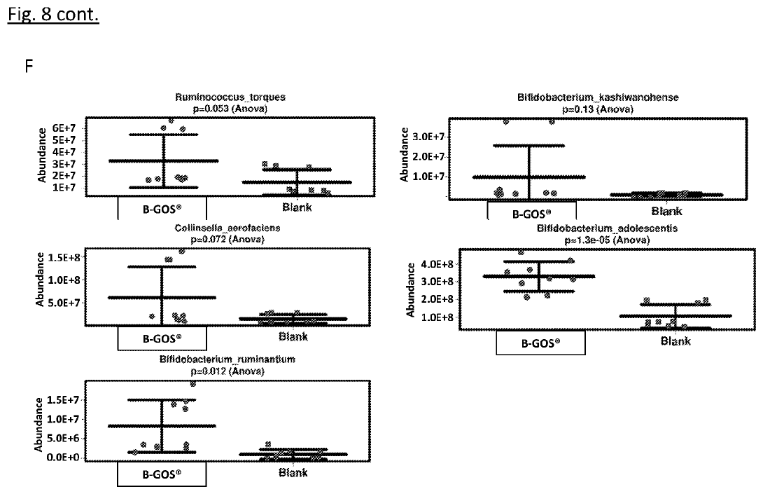

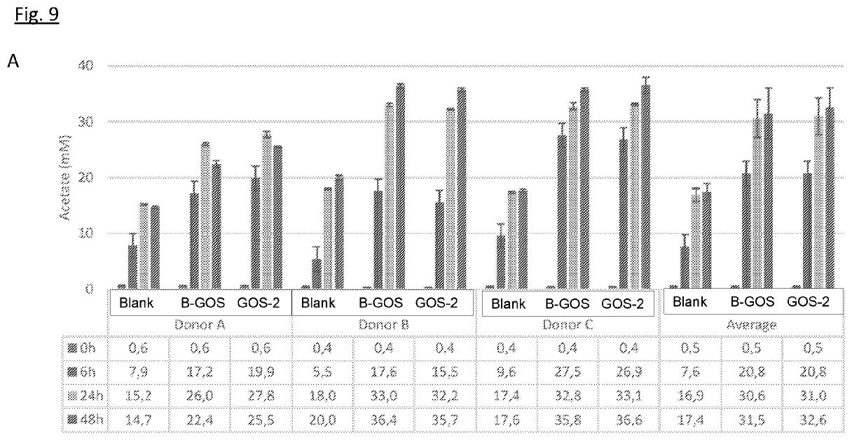

FIG. 8 are graphs showing changes in the microbial community composition in Example 4 (A) relative abundance (family level) 6 h after treatment, (B) linear discriminant analysis effect size of relative abundances (species level) 6 h after treatment, (C) boxplots for the most important enrichments (species level) 6 h after treatment, (D) absolute abundances (family level) 24 h after treatment, (E) linear discriminant analysis effect size of absolute abundances (species level) 24 h after treatment, (F) boxplots for the most important enrichments (species level) 24 h after treatment. *: p<0.05, **: p<0.01, ***: p<0.001. LDA=linear discriminant analysis.

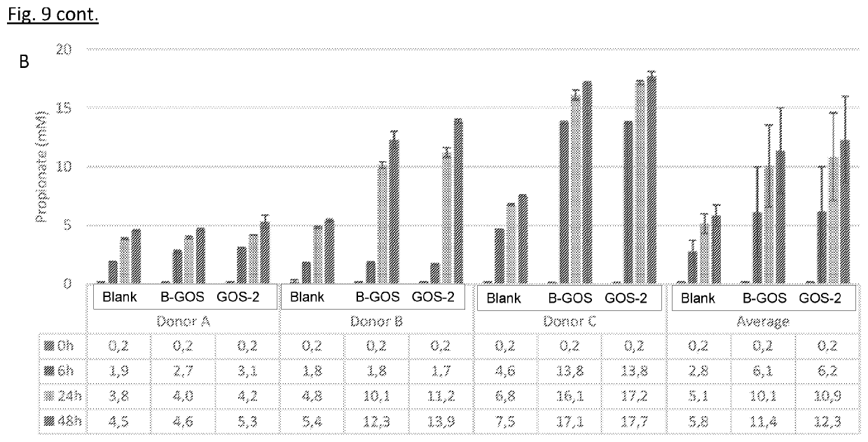

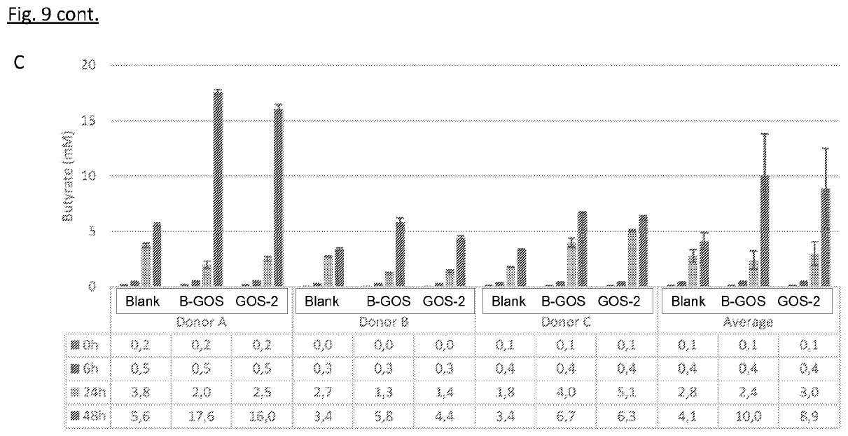

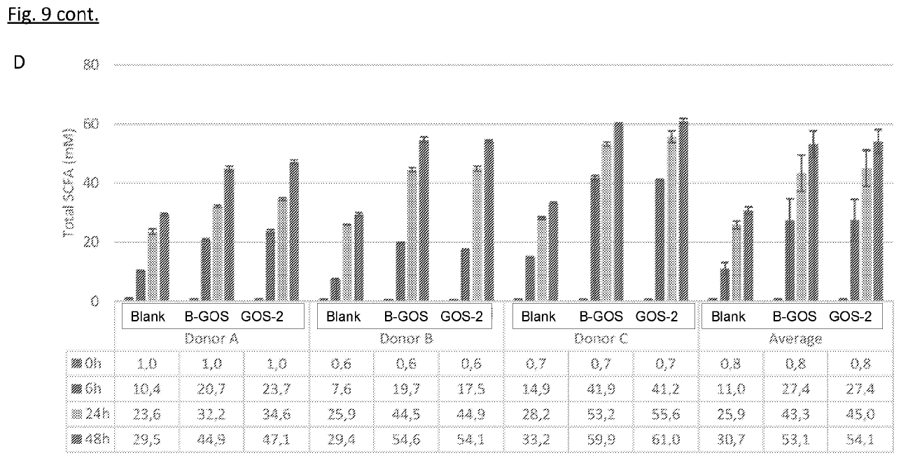

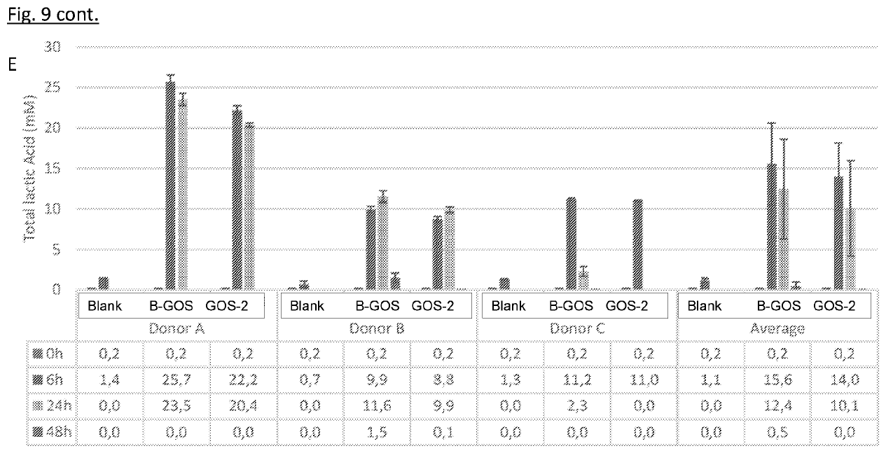

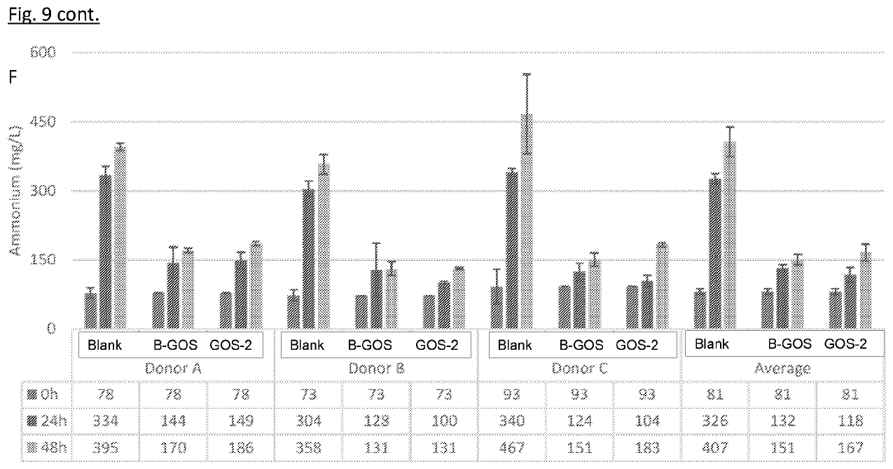

FIG. 9 are graphs showing microbial metabolic activity on different moments of the incubation in Example 5 (error bars reflect STDEV for individual donors and SEM for averages across donors) (A) acetate, (B) propionate, (C) butyrate, (D) total SCFA, (E) lactate and (F) ammonium, where two conditions (B-GOS and a commercially available GOS (denoted “GOS-2”)) were tested and a negative control (blank) was included.

### EXAMPLES

### Example 1—Oligosaccharide Syrup

A composition comprising oligosaccharide compounds according to the present invention, in the form of a syrup, was obtained by the following procedure. Lactose was rehydrated with potable water to give a working solution of between 35-65 wt % solids. The lactose solution was heat treated then cooled to 40-65 °C. The pH of the solution was adjusted to pH 5.5-7.5. A beta-galactosidase enzyme was then added to the solution in a closed vessel and subsequently allowed to react with the lactose to catalyse the transfer of galactose molecules to produce oligosaccharide compounds. The progress of the reaction was monitored by measuring the generation of glucose. The reaction was allowed to proceed for between 8 and 26 hours. The reaction was then terminated by high heat treatment. The reaction mixture was cooled and filtered by carbon filtration to remove the enzyme. The mixture was then dried by evaporation to reduce the water content to approximately 22-28 wt % to provide the product as a syrup.

### Example 2—Oligosaccharide Powder

A composition comprising oligosaccharide compounds according to the present invention, in the form of a powder, was obtained by a modification of the procedure described above. After removal of the enzyme, the mixture was further filtered to remove a significant portion of the glucose and other monosaccharides, reducing the monosaccharide content from around 23 wt % to around 5 wt %. The water content of the composition was then reduced by evaporation and the product dried to provide a powder having a water content to approximately 3-6 wt %.

### Example 3—Fractionation

Isolation of the different fractions (DP2, DP3, DP4 or DP5) from a sample of Example 2 was performed using a 5×70 cm BioGel P2 column, using water as eluent. The column was operated with a flow speed of 40 to 100 mL/h at room temperature (21 °C). Depending on the run, 0.5, 0.75, 1.0, 1.5 or 2.0 mL of a 0.5 g/mL solution of Example 2 in ultrapure water was loaded. The column was loaded in total 15 times with this solution to obtain enough material of the low abundant fractions, i.e. DP5. After a void volume of around 725 mL, 5 mL fractions were collected. Analysis using thin layer chromatography (TLC) and HPAEC-PAD were done to ensure molecules with the same DP are pooled.

Pooled fractions were frozen and lyophilized. The dry material of all runs was combined and redissolved. After a second cycle of freezing and lyophilization, the dry material was stored at 4 °C for further analyses or experimentation.

### Comparative Example 1

A commercially available composition comprising oligosaccharide compounds was obtained in powder form.

### Oligosaccharide Analysis

Samples of Example 2 of the present invention and Comparative Example 1 were analysed to determine their oligosaccharide content by the following procedure.

### Materials and Methods

Samples of dry powder of each of Example 2 and Comparative Example 1 were dissolved in water to provide solutions having a concentration of 40 g/I for analysis.

### Gel Permeation Chromatography

An HPLC apparatus equipped with a Rezex RSO and a RI detector and in-line desalting (for removal of salts and charged material like proteins) was used for the aqueous GPC separation of the components of the samples. The separation was performed at elevated temperature (80 °C). The separation range of the Rezex RSO column is from DP1 (monosaccharide) up to about DP10. All samples were analysed undiluted (at 40 g/L). Before analysis, all sample solutions were treated at 100 °C for ten minutes in order to remove any microbiological or enzymatic activity.

### GOS Fingerprinting

HPAEC-PAD (high performance anion exchange chromatography) equipped with a PA-1 column was used for separation of mono- and oligosaccharides of the different samples. Efforts were made to achieve a separation quality described in van Leeuwen et al., Carbohydrate Research 2016, 425, 48-58. A commercial maltooligosaccharide mixture and Comparative Example 1 were also injected for comparison of chromatograms with those reported by Van Leeuwen et al. Based on this, peak annotations were made for a number of peaks. The samples as used for GPC were 100-fold diluted with DMSO before injection.

### Results

Gel Permeation Chromatography (GPC-RI) Table 1 shows DP (degree of polymerisation) composition results for the samples using Rezex-RSO system, based on RI calibration with glucose (values expressed as g/L in the samples). All material eluting in the >DP5 window was combined.

TABLE 1

| | |
|---|---|
| Comp. Ex. 1 | Example 2 |

| Fraction | Conc. (g/l) | wt % | Conc. (g/l) | wt % |
|---|---|---|---|---|
| DP > 5 | 1.1 | 2.8 | 1.6 | 4.2 |
| DP = 5 | 2.2 | 5.6 | 2.6 | 6.9 |
| DP = 4 | 5.1 | 13.1 | 6.1 | 16.1 |
| DP = 3 | 10.7 | 27.4 | 12.6 | 33.2 |
| DP = 2 | 17.8 | 45.6 | 13.1 | 34.6 |
| glucose | 2.1 | 5.4 | 1.7 | 4.5 |
| galactose | 0.1 | 0.3 | 0.2 | 0.5 |
| Total | 39.0 | 100.0 | 37.9 | 100.0 |
| Total DP 2-5 | 35.7 | | 34.4 | |

The concentration information can be used to calculate the relative weight percentages of the different DP fractions of oligosaccharides contained in the samples, as shown in Table 1, wherein DP=2 refers to disaccharides, DP=3 refers to trisaccharides etc.

### GOS Fingerprinting

To identify the individual galactooligosaccharides in the sample (GOS fingerprinting) a gradient was developed giving comparable separation to that reported in van Leeuwen et al., Carbohydrate Research 2016, 425, 48-58. Peak annotations were made in the chromatograms of all GOS samples based on peak annotations made in van Leeuwen et al. for the oligosaccharide compounds. Retention windows of about 15 seconds were applied for peak annotation.

Table 2 shows information on HPAEC-PAD peak areas of all annotated peaks together with information on incubation conditions, sample concentration, dilution and injection volume as shown.

TABLE 2

| | |
|---|---|
| Comp. Ex. 1 | Example 2 |

| Compound ID | Peak areas | wt % | Peak areas | wt % |
|---|---|---|---|---|
| Unknown 1 | 1.6 | 0.6 | 0.5 | 0.2 |
| Unknown 2 | 1.5 | 0.5 | 1.9 | 0.7 |
| Galactose | 0.5 | 0.2 | 1.3 | 0.5 |
| Glucose | 19.5 | 7.0 | 17.5 | 6.7 |
| Unknown DP1 | 0.6 | 0.2 | 1.8 | 0.7 |
| Unknown 3 | 1.5 | 0.5 | 1.2 | 0.5 |
| 3 | 0.2 | 0.1 | 0.9 | 0.3 |
| Allolactose | 16.2 | 5.8 | 24 | 9.1 |
| Lactose | 48.4 | 17.5 | 30.9 | 11.8 |
| 6 | 7.2 | 2.6 | 2.1 | 0.8 |
| Unknown DP3 | 3.5 | 1.3 | 0.8 | 0.3 |
| 8a | 48.5 | 17.5 | 38.5 | 14.7 |
| 8b | 18.1 | 6.5 | 14.6 | 5.6 |
| 9 | 7.9 | 2.9 | 8.1 | 3.1 |
| 10 | 16 | 5.8 | 13.1 | 5.0 |
| 11 | 27.6 | 10.0 | 20.4 | 7.8 |
| 12 | 1.4 | 0.5 | 28.9 | 11.0 |
| 13 | 20.4 | 7.4 | 4.3 | 1.6 |
| 17 | 11.8 | 4.3 | 2.7 | 1.0 |
| 18 | 7.1 | 2.6 | 0.9 | 0.3 |
| 22 | 6.4 | 2.3 | 3.9 | 1.5 |
| 23 | 1.9 | 0.7 | 4.6 | 1.8 |
| 24 | 1.1 | 0.4 | 2 | 0.8 |
| 29 | 4.7 | 1.7 | 12.8 | 4.9 |
| 30 | 2.6 | 0.9 | 12.6 | 4.8 |
| 31 | 0.8 | 0.3 | 12.3 | 4.7 |
| sum | 277.1 | 100.0 | 262.5 | 100.0 |

The peak areas were assumed to approximately correspond to the amount of each oligosaccharide compound present in the composition. Where a particular oligosaccharide compound was not identified then “unknown” and a number is entered in the table. The identified compounds are either identified by name or by a number which corresponds to the number assigned to particular galactooligosaccharides in van Leeuwen et al., Carbohydrate Research 2016, 425, 48-58. A list of these galactooligosaccharides and their corresponding numbers is provided below.

The oligosaccharide components (a)-(e) discussed above, wherein each X group is Glu, correspond to the following numbered entries in Table 2 above:
(a) D-Gal-(β1-3)-D-Gal-(β1-4)-D-Glu-12
(b) D-Gal-(β1-3)-D-Gal-(β1-3)-D-Glu-29
(c) D-Gal-(β1-3)-D-Gal-(β1-2)-D-Glu-30
(d) D-Gal-(β1-3)-D-Gal-(β1-3)-D-Gal-(β1-4)-D-Glu-31
(e) D-Gal-(β1-4)-D-Gal-(β1-4)-D-Glu-11.

### Prebiotic Effects—Butyrate Production

Butyrate is produced by gut microbiota which convert acetate and/or lactate (along with other substrates) to butyrate. As butyrate can be a secondary metabolite, it is often produced during late stages of fermentation as butyrate can be directly produced by certain bacterial butyrate producers.

These experiments were aimed at assessing the difference in butyrate production of the compositions of the present invention alongside a comparative GOS composition so as to see if they were more effective as a prebiotic. The experiments used the following procedure.

The compositions of Example 2 of the present invention, Comparative Example 1 and a control blank sample were subjected to dialysis using a 0.5 kDa membrane to provide 5 g/I samples which were then mixed with faecal matter obtained from three healthy human adult subjects (donors A, B and C). The mixtures were shaken under anaerobic conditions and monitored over a 48 hour period for colonic fermentation products including butyrate (with 6, 24 and 48 h collection points). The distribution of oligosaccharides in the mixtures was also monitored over this time period using the method described above in relation to Table 2. The results show that Example 2 was well fermented by all donors, mainly during the time period 0-24 hours and that butyrate production increased compared to the Comparative Example 1 and the control at the 6 and 48 hour time points. The results of the butyrate analysis are shown in FIG. 1. The results of the oligosaccharides analysis for the samples at the different time points are shown in Table 3. These results show that the oligosaccharides in the samples were actively consumed by the microbiota present in the faecal samples during the experiments.

To assess whether treatment effects on gut microbial activity were statistically significant, three two-sided T-tests were performed between Example 2 and the control, Comparative Example 1 and the control and Example 2 and Comparative Example 1 to obtain p-values. The Benjamini-Hochberg false discovery rate (FDR) was also used in this analysis. Differences between treatment effects were considered significant when the obtained p-value was smaller than a reference value. Table 4 below shows the differences in the averaged butyrate production for the compared samples over 48 hours and the asterisk denotes whether the difference was considered significant according to the analysis described above. These results show that the increase in butyrate production provided by Example 2 during the time period 0-48 hours was statistically significant compared to the control and Comparative Example 1.

TABLE 3

| | Comp. Ex. 1 | | | | | | | | | | Example 2 | | | | | | | | | |
|---|---|---|---|---|---|---|---|---|---|---|---|---|---|---|---|---|---|---|---|---|
| | Peak areas | | | | | | | | | | Peak areas | | | | | | | | | |
| | | 6 h | | | 24 h | | | 48 h | | | 0 h | 6 h | | | 24 h | | | 48 h | | |
| | | Donor | | | | | | | | | | | | | | | | | | |
| Cpd. ID | 0 h | A | B | C | A | B | C | A | B | C | A | A | B | C | A | B | C | A | B | C |
| Unknown 1 | 1.6 | 0.2 | 0.8 | 0.1 | 0.0 | 0.0 | 0.0 | 0.0 | 0.1 | 0.2 | 0.5 | 0.7 | 0.5 | 0.1 | 0.0 | 0.0 | 0.0 | 0.0 | 0.0 | 0.1 |
| Unknown 2 | 1.5 | 0.1 | 0.0 | 0.0 | 0.0 | 0.0 | 0.0 | 0.0 | 0.0 | 0.0 | 1.9 | 0.4 | 0.3 | 0.0 | 0.0 | 0.3 | 0.0 | 0.0 | 0.0 | 0.0 |
| Galactose | 0.5 | 2.4 | 45.1 | 0.7 | 0.0 | 0.1 | 0.0 | 0.0 | 0.0 | 0.0 | 1.3 | 2.1 | 47.3 | 0.8 | 0.0 | 0.1 | 0.0 | 0.0 | 0.0 | 0.0 |
| Glucose | 19.5 | 2.9 | 34.1 | 0.2 | 0.0 | 0.1 | 0.0 | 0.0 | 0.0 | 0.0 | 17.5 | 2.6 | 26.9 | 0.3 | 0.0 | 0.0 | 0.0 | 0.0 | 0.0 | 0.0 |
| Unknown DP1 | 0.6 | 0.4 | 1.7 | 0.0 | 0.0 | 0.0 | 0.0 | 0.0 | 0.0 | 0.0 | 1.8 | 0.5 | 1.6 | 0.2 | 0.3 | 0.0 | 0.0 | 0.0 | 0.0 | 0.0 |
| Unknown 3 | 1.5 | 0.1 | 0.6 | 0.1 | 0.1 | 0.2 | 0.0 | 0.0 | 0.1 | 0.0 | 1.2 | 0.0 | 0.0 | 0.1 | 0.3 | 0.0 | 0.0 | 0.0 | 0.0 | 0.0 |
| 3 | 0.2 | 0.2 | 1.2 | 0.4 | 0.0 | 0.2 | 0.0 | 0.0 | 0.3 | 0.1 | 0.9 | 0.3 | 1.7 | 0.4 | 0.0 | 0.2 | 0.0 | 0.0 | 0.0 | 0.3 |
| Allolactose | 16.2 | 0.0 | 0.0 | 0.0 | 0.0 | 0.0 | 0.0 | 0.0 | 0.0 | 0.0 | 24 | 1.3 | 0.0 | 0.0 | 0.0 | 0.0 | 0.0 | 0.0 | 0.0 | 0.0 |
| Lactose | 48.4 | 0.7 | 1.9 | 0.4 | 0.4 | 0.5 | 0.4 | 0.3 | 0.5 | 0.3 | 30.9 | 0.3 | 5.3 | 0.6 | 0.3 | 0.4 | 0.4 | 0.3 | 0.5 | 0.3 |
| 6 | 7.2 | 5.6 | 4.2 | 2.1 | 0.1 | 0.1 | 0.2 | 0.2 | 0.2 | 0.1 | 2.1 | 1.9 | 2.6 | 0.5 | 0.2 | 0.1 | 0.2 | 0.2 | 0.2 | 0.1 |
| Unknown DP3 | 3.5 | 0.4 | 2.8 | 0.1 | 0.1 | 0.0 | 0.0 | 0.0 | 0.0 | 0.0 | 0.8 | 0.1 | 1.3 | 0.1 | 0.0 | 0.0 | 0.0 | 0.0 | 0.0 | 0.0 |
| 8a | 48.5 | 0.7 | 10.2 | 0.5 | 0.4 | 0.1 | 0.1 | 0.0 | 0.1 | 0.0 | 38.5 | 1.6 | 17.0 | 0.6 | 0.0 | 0.1 | 0.0 | 0.0 | 0.0 | 0.1 |
| 8b (**) | 18.1 | 0.4 | 0.0 | 0.0 | 0.1 | 0.3 | 0.2 | 0.1 | 0.1 | 0.0 | 14.6 | 0.3 | 0.0 | 0.0 | 0.0 | 0.2 | 0.1 | 0.1 | 0.1 | 0.1 |
| 9 | 7.9 | 1.1 | 1.5 | 1.6 | 0.0 | 1.2 | 0.7 | 0.0 | 0.8 | 0.0 | 8.1 | 4.3 | 3.7 | 2.3 | 0.0 | 0.9 | 0.6 | 0.1 | 1.0 | 0.0 |
| 10 | 16 | 2.6 | 4.3 | 2.1 | 1.7 | 2.0 | 1.6 | 1.7 | 2.0 | 1.3 | 13.1 | 3.9 | 4.9 | 2.1 | 1.2 | 1.6 | 1.4 | 1.7 | 1.9 | 1.7 |
| 11 | 27.6 | 1.9 | 5.7 | 1.6 | 1.9 | 2.0 | 0.7 | 0.0 | 1.9 | 0.2 | 20.4 | 3.2 | 4.5 | 1.6 | 1.2 | 1.7 | 0.5 | 0.3 | 2.6 | 0.6 |
| 12 | 1.4 | 1.4 | 0.8 | 0.9 | 0.4 | 0.4 | 0.4 | 0.1 | 1.1 | 0.1 | 28.9 | 1.1 | 5.4 | 1.2 | 0.4 | 0.7 | 0.3 | 0.3 | 0.7 | 0.2 |
| 13 | 20.4 | 0.2 | 4.1 | 0.5 | 0.7 | 0.8 | 0.3 | 0.1 | 0.9 | 0.1 | 4.3 | 1.1 | 2.0 | 0.6 | 0.2 | 0.6 | 0.2 | 0.3 | 0.4 | 0.3 |
| 17 | 11.8 | 0.5 | 4.7 | 1.1 | 0.5 | 0.4 | 0.1 | 0.1 | 0.6 | 0.1 | 2.7 | 1.1 | 1.2 | 0.6 | 0.0 | 0.2 | 0.1 | 0.3 | 0.5 | 0.3 |
| 18 | 7.1 | 1.3 | 4.7 | 0.9 | 1.3 | 1.9 | 0.8 | 0.0 | 2.4 | 0.5 | 0.9 | 2.0 | 1.8 | 0.9 | 0.9 | 1.4 | 0.6 | 0.5 | 1.4 | 0.5 |
| 22 | 6.4 | 0.5 | 2.7 | 0.9 | 0.9 | 0.7 | 0.7 | 0.4 | 0.8 | 0.7 | 3.9 | 0.0 | 1.0 | 0.6 | 0.0 | 0.6 | 0.5 | 0.8 | 0.5 | 0.7 |
| 23 | 1.9 | 0.4 | 1.3 | 1.3 | 1.0 | 1.1 | 0.7 | 0.7 | 1.5 | 0.9 | 4.6 | 1.7 | 1.9 | 1.1 | 0.4 | 0.6 | 0.8 | 0.9 | 1.3 | 1.0 |
| 24 | 1.1 | 0.0 | 1.1 | 0.8 | 0.8 | 0.3 | 0.3 | 0.4 | 0.5 | 0.5 | 2.0 | 0.8 | 1.1 | 0.6 | 0.4 | 0.3 | 0.2 | 0.5 | 0.9 | 0.5 |
| 29 | 4.7 | 0.1 | 2.0 | 1.0 | 0.2 | 0.4 | 0.0 | 0.0 | 0.3 | 0.0 | 12.8 | 1.1 | 2.0 | 0.4 | 0.0 | 0.0 | 0.0 | 0.1 | 0.4 | 0.1 |
| 30 | 2.6 | 0.0 | 1.3 | 0.3 | 0.0 | 0.5 | 0.0 | 0.0 | 0.6 | 0.0 | 12.6 | 0.0 | 1.8 | 0.9 | 0.0 | 0.5 | 0.0 | 0.1 | 0.0 | 0.2 |
| 31 | 0.8 | 0.0 | 0.5 | 0.3 | 0.5 | 0.5 | 0.0 | 0.0 | 0.4 | 0.0 | 12.3 | 0.0 | 2.0 | 0.3 | 0.2 | 0.3 | 0.0 | 0.0 | 0.0 | 0.0 |
| sum | 277.1 | 24.1 | 137.3 | 18.1 | 11.1 | 13.7 | 7.1 | 4.1 | 15.1 | 5.2 | 262.5 | 32.4 | 137.8 | 16.9 | 6.1 | 10.7 | 5.8 | 6.6 | 12.3 | 7.0 |

TABLE 4

| | Example 2 - | Comp. Ex. 1 - | Example 2 - |
|---|---|---|---|
| D 0-48 h / Production | Control | Control | Comp. Ex. 1 |
| Butyrate (mM) | 5.88* | 4.75* | 1.13* |

In summary, the present invention provides a composition comprising oligosaccharide compounds, for example galactooligosaccharide compounds, which includes: (a) at least 8 wt % Gal-(β1-3)-Gal-(β1-4)-X^{a}; (b) at least 3 wt % Gal-(β1-3)-Gal-(β1-3)-X^{b}; and (c) at least 5 wt % Gal-(β1-3)-Gal-(β1-2)-X^{c}, based on the total weight of oligosaccharide compounds present in the composition; wherein X^{a}, X^{b} and X^{c} are each independently selected from monosaccharides. These compositions contain relatively high amounts of the oligosaccharide compounds (a), (b) and (c) and a relatively high amount of β1-3 Gal-Gal linkages, compared to known oligosaccharide compositions. Advantageously, the compounds present in the composition showed a belated effect of butyrate increase over time which is believed to be due to the cross-feeding of other bacteria which are acetate-producers and this acetate in turn stimulates butyrate producers to growth and secrete butyrate. These particular features of the composition are believed to provide benefits to the gut health of a consumer, for example due to these compositions providing an increased production of butyrate in the gut of a consumer compared to known compositions.

### Example 4—Assessment of (GOS) for the Reduction of Ammonium and pH in a Short-Term Colonic Fermentation Model

Experiments were conducted in order to evaluate the influence of GOS of the present invention (denoted B-GOS) on the composition and activity of the colonic microbiota using short-term colonic incubations. Evaluation was based on the effects on overall microbial fermentation (pH, gas production), microbial metabolic activity (production of SCFA, lactate, and ammonium), and community composition using shallow shotgun sequencing. In addition, B-GOS utilization by gut microbiota was analyzed to understand the dynamics of this process.

### Fecal Samples

Following the collection of fecal material from three healthy adult donors, fecal suspensions were prepared and mixed with a cryoprotectant. Suspensions were then aliquoted, flash frozen, and stored at −80 °C until needed.

### Product Dialysis

The GOS composition of the present invention (herein also referred to as B-GOS”) was dialyzed prior to colonic incubation to simulate absorption during small intestinal passage. Stock solutions of B-GOS were prepared in water at 40 g/L which were then added inside dialysis membranes (0.5 kDa pore size) to allow for monosaccharides and disaccharides to pass through the membrane. Membranes were sealed and the stock solution was dialyzed in a solution of NaHCO₃ (3.75 g/L, pH 7.0) for 24 h at a low temperature to prevent microbial growth.

### Short-Term Colonic Incubations

Short-term colonic incubations were performed as previously described in Van den Abbeele, et. al. 2018 (Different oat ingredients stimulate specific microbial metabolites in the gut microbiome of three human individuals in vitro. ACS Omega 2018, 3, 12446-12456). Briefly, individual reactors were filled with sugar-depleted nutritional medium containing basal colonic nutrients. Next, dialyzed B-GOS (5 g/L final concentration) or blank medium was added followed by fecal inoculum. Incubations were performed in triplicate for B-GOS and blank (media control) and for each donor (six incubations per donor, three with B-GOS and three blank). Samples were collected at 0 h, 6 h, 24 h, and 48 h. Shallow shotgun sequencing and flow cytometry (cell counts) were performed on samples collected at 6 h and 24 h.

### Microbial Metabolic Activity Analysis

Change in pH, gas pressure, SCFAs, lactate and ammonium were measured at 0 h, 6 h, 24 h, and 48 h. Changes in pH were measured using a Senseline F410 pH meter (ProSense, Oosterhout, The Netherlands). Changes in gas pressure were measured using a hand-held pressure indicator (CPH6200; Wika, Echt, The Netherlands). Acetate, propionate and butyrate were measured as previously described by De Weirdt et al. 2010 (Human faecal microbiota display variable patterns of glycerol metabolism. FEMS Microbiol Ecol 2010, 74, 601-611). Lactate levels were monitored using a commercially available enzymatic assay kit (R-Biopharm, Darmstadt, Germany) according to the manufacturer's instructions. Ammonium was analyzed as described by Van de Wiele et al. 2004 (Prebiotic effects of chicory inulin in the simulator of the human intestinal microbial ecosystem. FEMS Microbiol Ecol 2004, 51, 143-153).

### Microbial Community Analysis

DNA libraries were prepared using the Illumina Nextera XT library preparation kit, with a modified protocol. Library quantity was assessed with Qubit (ThermoFisher). Libraries were then sequenced on an Illumina HiSeq platform 2×150 bp. Unassembled sequencing reads were directly analyzed according to Ottensen et al. 2016 (Enrichment dynamics of Listeria monocytogenes and the associated microbiome from naturally contaminated ice cream linked to a listeriosis outbreak. BMC Microbiol 2016, 16, 275), Ponnusamy et al. 2016 (Cross-talk among flesh-eating Aeromonas hydrophila strains in mixed infection leading to necrotizing fasciitis. Proc Natl Acad Sci USA 2016, 113, 722-727), Hasan et al. 2014 (Microbial community profiling of human saliva using shotgun metagenomic sequencing. PLoS One 2014, 9, e97699), and Lax et al. 2014 (Longitudinal analysis of microbial interaction between humans and the indoor environment. Science 2014, 345, 1048-1052) for multi-kingdom microbiome analysis and quantification of relative abundances. Briefly, curated genome databases were utilized in combination with a high-performance data-mining algorithm that rapidly disambiguates hundreds of millions of metagenomic sequence reads into the discrete microorganisms engendering the sequences. The total number of bacterial cells was determined using a BD FACSVerse Cell Analyzer (BD Biosciences, Franklin Lakes, NJ, USA) on the high flow rate setting with a threshold of 200 on the SYTO channel. Proportional values obtained using shotgun sequencing were converted to absolute quantities by multiplying relative abundances of each population in a sample with the total cell count obtained by flow cytometry.

### GOS Utilization by Gut Microbiota Overtime

GOS chain length distribution analysis was performed using undialed B-GOS, dialyzed B-GOS and samples obtained at all fermentation timepoints (6, 24 and 48 hours). B-GOS chain length distribution was performed using gel permeation chromatography (GPC).

### Gel Permeation Chromatography

A HPLC equipped with a Rezex RSO and a RI detector and in-line desalting (for removal of salts and charged material like proteins) was applied for the aqueous GPC separation. The separation was performed at elevated temperature (80 °C). The separation range of the Rezex RSO column was ranging from DP1 (monosaccharide) up to about DP10.

### Statistical Methods

Study outcomes between B-GOS treatment and blank were calculated using paired two-sided student's t-tests. To control the proportion of false discoveries when conducting a high number of comparisons, the Benjamini-Hochberg false discovery rate (FDR) was applied. Differences between treatment effects were considered significant when the obtained p-value (obtained through the paired two-sided t-test) was smaller than the reference value. The reference value was obtained by ranking the obtained p-values in ascending order within the donor group. The rank of a given p-value was termed (i) and varied between 1 to the total amount of p-values (m=3). The reference value was calculated by multiplying the FDR with the rank of the p-value, divided by the total amount of comparisons made (ref=FDR*i/m). To compare treatment effects in terms of changes in pH, gas pressures, and microbial metabolite production (SCFA, lactate, and ammonium), the FDR was set at 0.10, meaning that the lowest p-value should be below 0.033 to be significantly different, the second lowest below 0.066, etc. Comparisons of the absolute and relative abundances of specific members of the microbial community were conducted using analysis of variance (ANOVA). Linear discriminant analysis (LDA) effect size analysis (LEfSe) was conducted to detect between group differences in bacterial abundances. A p-value of <0.05 was considered statistically significant. All statistical analyses were performed using Microsoft Excel version 2110 (Microsoft, Redmond, WA, USA). Principle coordinates analysis (PCoA) was performed using Analyze-it (v4.51) software. The increase or decrease at 6 h or 24 h of incubation for each parameter was used to create a joint PCoA biplot for each of the two timepoints.

### Microbial Metabolic Activity

The pH remained relatively stable at all timepoints in the blank (untreated) reactors and was reduced relative to 0 h at 6 h, 24 h, and 48 h with B-GOS treatment (FIG. 2A). For the blank samples, gas production was somewhat higher between 6 h and 24 h than between 0 h and 6 h, and decreased rather dramatically between 24 h and 48 h (FIG. 2B). A similar pattern was observed for B-GOS, though the gas production was greater overall. pH and gas production for the individual donors are shown in FIGS. 3A and 3B, respectively.

Average changes in SCFA levels are shown in FIG. 4D and data for individual donors is shown in FIG. 5D. Acetate levels increased between 0 h and 6 h and between 6 h and 24 h, and then remained stable between 24 h and 48 h in the blank reactors (FIG. 4A). In the reactors treated with B-GOS, the most dramatic production of acetate occurred between 0 h and 6 h, with a comparatively reduced level between 6 h and 24 h and having the least production between 24 h and 48 h. Data for individual donors is shown in FIG. 4. The average propionate data showed that production was increased in the presence of B-GOS relative to the blank, and production was greatest between 0 h and 6 h, followed by between 6 h and 24 h, and was lowest between 24 h and 48 h (FIG. 4B). Propionate production varied between donors (FIG. 5B); little propionate was produced with Donor A and the levels were similar between treated and blank. Butyrate production showed a different pattern, with the lowest production between 0 h and 6 h and the highest production between 24 h and 48 h with B-GOS treatment (FIG. 4C). While Donors B and C produced a similar total amount of butyrate with B-GOS (5.7 mM and 6.6 mM, respectively), the total amount produced by DonorA was comparatively much higher (17.4 mM in total) (FIG. 5C). Total SCFA production is shown in FIG. 4D. SCFA production was higher in the B-GOS treated reactors compared with the blank reactors. In general, SCFA production was greatest between 0 h and 6 h, followed by between 6 h and 24 h; the least production was observed between 24 h and 48 h. Data for individual donors is shown in FIG. 5D. Lactic acid levels were highest between 0 h and 6 h and the level was higher with B-GOS treatment versus blank (FIG. 4E). Though there were some differences between donors (FIG. 5E), lactic acid levels were reduced between 6 h and 24 h and between 24 h and 48 h. With B-GOS treatment, the level of lactic acid decrease between 24 h and 48 h was greater than blank, though this was less pronounced for Donor C. Treatment with B-GOS reduced the production of ammonium for all donors (FIG. 4F); there were no obvious differences among the donors (FIG. 5F).

### Microbial Community Composition

The microbial community composition at the start of the study is shown for each donor in FIG. 6. While the number of cells/g fecal matter varied among the donors, Bacteroidetes were most abundant, followed by Firmicutes and Actinobacteria. The other phyla made up a very small proportion of the community composition. PCoA plots showing relative data at 6 h (FIG. 7A) and absolute data at 24 h (FIG. 7B) demonstrate clear shifts in microbial community composition between B-GOS and blank reactors for each of the donors as well as differences among donors.

Average relative abundances for the microbial community composition at the family level at 6 h are shown in FIG. 8A. Treatment with B-GOS resulted in a significant decrease in the relative abundance for several families. LEfSe revealed a high LDA score of >4 for Bifidobacterium longum with B-GOS treatment at 6 h (relative abundance) (FIG. 8B). The relative abundances of B. longum and Megamonas (unspecified) were significantly increased with B-GOS treatment compared with blank at 6 h (p=0.006 and p=0.03, respectively [ANOVA]) (FIG. 8C). Changes in absolute abundances at the family level at 24 h are shown in FIG. 8D. Compared with blank, treatment with B-GOS resulted in significant changes for several families, most notably, there was a significant increase in the absolute abundance of Bifidobacteriaceae (p<0.001). Lactobacillaceae were also significantly increased (p<0.05) and Clostridiales (p<0.001), Erysipelotrichaceae (p<0.05), Odoribacteraceae (p<0.01), and Oscillospiraceae (p<0.001) were significantly decreased with B-GOS treatment versus blank. Absolute abundances at the family level at 24 h are shown in Table 5 below (according to donor and overall) (In Table 5, the intensity of shading is a measure for the abundance of a family in the different conditions per donor/average. Values in bold represent a statistically significant difference in abundance between blank and B-GOS using unpaired t-tests on technical replicates (n=3) for within donor comparisons and using paired t-tests for across donor comparisons).

The absolute abundance of Streptococcaceae increased significantly with B-GOS relative to blank for donor A but was below the limit of quantification for the other two donors. LEfSe revealed a high LDA score of >4 for Bifidobacterium adolescentis, Collinsella spp., and Collinsella aerofaciens and of >3 (but <4) for Ruminococcus torques, Bifidobacterium kashiwanohense, and Bifidobacterium ruminantium with B-GOS treatment at 24 h (absolute abundance) (FIG. 8E). The absolute abundances of B. adolescentis and B. ruminantium were significantly increased with B-GOS treatment compared with blank at 24 h (p<0.001 and p=0.01, respectively [ANOVA]) (FIG. 8F).

GOS utilization by gut microbiota Using short-term colonic incubations, the effects of B-GOS on the overall microbial fermentation was assessed along with microbial metabolic activity, and microbial community composition of colonic bacteria isolated from healthy donors. B-GOS had notable effects on these parameters, including increased SCFA production and increased abundance of beneficial bacteria. It has been also noted that almost 95% B-GOS was taken up by gut microbiota already by 24 hours.

During the experiments, it was found that B-GOS increased the production of acetate, propionate, butyrate, and lactate. This result agrees with previous in vitro studies using fecal batch cultures that reported an increased production of acetate with B-GOS supplementation, an increase in acetate and lactate with B-GOS supplementation, and an increased production of lactate and all SCFAs, particularly butyrate, with B-GOS supplementation. The increased butyrate production was quite marked in our study and could be observed as early as 6 h to 24 h, demonstrating a maximum increase at 24 h to 48 h. The marked increase in butyrate production was likely largely driven by lactate-to-butyrate conversion, which would explain the consumption of lactic acid after 6 h. Acetate-to-butyrate conversion may also have contributed to the increase in butyrate. Butyrate is preferentially consumed by colonocytes, making it an important component in gut membrane health. Additionally, butyrate plays an important role in regulating the integrity of the epithelial barrier via coordinated regulation of tight junction proteins. The importance of this function is highlighted by the fact that loss of barrier integrity is thought to contribute to metabolic disorders, inflammatory bowel disease, and obesity. In obese adults, B-GOS supplementation was shown to improve intestinal barrier function. During the experiments, B-GOS stimulated health-related microbial metabolites, confirming its usefulness as a prebiotic, and, unexpectedly, demonstrating its beneficial effects on unfavorable metabolites such as ammonium.

With B-GOS supplementation, the production of ammonium was reduced relative to the blank. As ammonium is a marker of proteolytic fermentation, a reduction indicates that the level of proteolytic fermentation was low with B-GOS, indicating a shift in the fermentation pattern to one more beneficial for the host. This may be considered beneficial, as some metabolic derivatives of proteolytic fermentation are implicated in disease, including colorectal cancer. Avoiding proteolytic fermentation is considered beneficial, as highly toxic compounds may be produced during this process. This is especially important for individuals on a high protein diet due to increased protein loads and increased proteolytic fermentation.

A previous in vitro study reported that B. longum and other Bifidobacterium spp. along with several Lactobacillus spp. were directly involved in B-GOS fermentation. In our experiments, the relative abundance of B. longum and Megamonas (unspecified) was significantly increased (versus blank) at the 6 h timepoint after B-GOS supplementation. In addition, at 24 h the absolute abundance of B. adolescentis and B. ruminantium was significantly increased (versus blank) as was the absolute abundance of Bifidobacteriaceae and Lactobacillaceae. Other in vitro fecal batch studies have shown that B-GOS supplementation increases the growth of bifidobacteria and lactobacilli and studies in humans have demonstrated that B-GOS supplementation results increased bifidobacteria and lactobacilli in healthy adults and elderly. Bifidobacteria and lactobacilli are well known for their role in human health. The increase in both was likely responsible for augmenting the observed increase in butyrate production via lactate production. Thus, the observed increase in bifidobacteria and Lactobacillaceae support the prebiotic effects of B-GOS. A role for Megamonas in health or disease has not been clearly defined, so it is difficult to speculate on the implications of this finding.

There were some donor specific effects regarding propionate and butyrate production. Donor A produced very little propionate and there was no difference in propionate production between blank or B-GOS supplementation. However, this donor produced a much higher amount of butyrate with B-GOS supplementation than the other two donors. In addition, lactate production and consumption occurred to a greater degree in Donor A than the others. We observed an enrichment of Streptococcaceae for Donor A upon B-GOS supplementation. This may have contributed to the early strong increase in lactate production (0-6 h) and subsequent conversion into butyrate (24-48 h). Interestingly, Donor A and Donor B had an increase in the absolute abundance of both Bifidobacteriaceae and Lactobacillaceae while Donor C had an increase in Bifidobacteriaceae but not Lactobacillaceae. However, these differences in the effect of B-GOS on the three donors do not completely explain the differences between donors in butyrate or lactate production.

In conclusion, B-GOS demonstrated prebiotic activity in short-term colonic incubations using the colonic microbiota of three healthy adult donors. Supplementation resulted in an increase in lactic acid and SCFA production, including butyrate, and increased growth of the beneficial bacterial families, Bifidobacteriaceae and Lactobacillaceae, while for the first time it was demonstrated that B-GOS reduced unfavorable metabolites such as ammonium.

### Example 5—Comparative Analysis of the Effect of B-GOS with Commercially Available GOS for the Reduction of Ammonium and pH in a Short-Term Colonic Fermentation Model

Experiments were conducted to evaluate the influence of a powdered version of the GOS of the present invention (denoted “B-GOS”) and a commercially available GOS Powder (denoted “GOS-2”) on the colonic microbiota of three healthy human adults. Evaluation was based on the effects on overall microbial carbohydrate fermentation (acetate, propionate and butyrate) and microbial metabolic activity (production of SCFA, lactate and ammonium). To do so, short-term colonic incubations were performed in a similar manner to those outlined in Example 4 above and the results subjected to the same statistical analysis.

### Carbohydrate Fermentation

As shown in FIG. 9A, stimulatory effects started between 0-6 h, with both products yielding similar acetate concentrations after 6 h. By the end of the incubation, both products had yielded similar acetate concentrations, significantly higher than the blank. Consumption of acetate in the incubations with donor A (24-48 h) may have been attributed to conversion into butyrate, which would explain the stimulatory effects on butyrate- and gas-production during the same timeframe.

FIG. 9B shows that both products yielded significantly higher propionate levels than the blank (strongest effects in donors B and C). GOS-2 yielded slightly higher propionate concentrations than B-GOS. Fermentation in donor C was characterized by strong propionate production between 0-6 h, in donor B by strong propionate production between 6-24 h.

In FIG. 9C, it can be seen that by the end of the incubation, both products had significantly stimulated butyrate production across the three donors, with Bimuno resulting in higher butyrate concentrations than GOS-2. Butyrate-production between 24-48 h in donor A may have been attributed to conversion of acetate (and lactate, see below) within the same timeframe.

### Microbial Metabolic Activity

Total SCFA levels are reflective of the overall fermentation of test ingredients. Both products yielded significantly higher SCFA levels than the blanks (FIG. 9D). Stimulatory effects on SCFA started between 0-6 h, with both products yielding similar SCFA concentrations after 6 h. This reflects efficient product fermentation by the gut microbiota. By the end of the incubation, both products had yielded similar SCFA concentrations, significantly higher than the blank.

As shown in FIG. 9E, production and consumption of lactate were low in the blank incubations. Each product significantly stimulated the production of lactate between 0-6 h, another confirmation of efficient product fermentation. The strongest stimulatory effect was obtained with Bimuno. Lactate accumulation (observed after 6 h) implies that the lactate production rate was higher than the lactate consumption rate. Overall, lactate produced during the first 6 h was efficiently consumed by the end of the incubation in all conditions, which is indicative of efficient lactate conversion (into propionate/butyrate).

As shown in FIG. 9F, both products significantly reduced the production of ammonium over the course of the incubation. Reduction was observed already after 24 h. The strongest reduction was obtained with B-GOS, which yielded significantly lower ammonium levels after 48 h than GOS-2.

### CONCLUSIONS

The aim of the experiments was to investigate the potential health-promoting effects of the B-GOS composition of the present invention alongside a commercially available GOS Powder (GOS-2). In order to do so, effects on microbial metabolic activity were studied, using the gut microbiota of three adult donors.

The short-term colonic incubations illustrated that both products were fermented by the gut microbiota of the three donors, stimulating production of acetate, propionate, butyrate and lactate, and significantly lowering production of ammonium. Both products yielded similar levels of acetate. B-GOS yielded higher butyrate- and lactate-levels; GOS-2 yielded higher propionate-levels.

Ammonium production results from proteolytic microbial activity, which is associated with formation of toxic by-products such as p-cresol. Therefore, high ammonium production in the colon has been associated with detrimental health effects. As a consequence, products that reduce ammonium production are health-beneficial and the GOS of the present invention reduces the formation of toxic by-products and represent a useful medicament or nutritional supplement conferring superior results over current commercially available GOS products.

Although a few preferred embodiments have been shown and described, it will be appreciated by those skilled in the art that various changes and modifications might be made without departing from the scope of the invention, as defined in the appended claims.

Throughout this specification, the term “comprising” or “comprises” means including the component(s) specified but not to the exclusion of the presence of other components. The term “consisting essentially of” or “consists essentially of” means including the components specified but excluding other components except for materials present as impurities, unavoidable materials present as a result of processes used to provide the components, and components added for a purpose other than achieving the technical effect of the invention. Typically, when referring to compositions, a composition consisting essentially of a set of components will comprise less than 5% by weight, typically less than 3% by weight, more typically less than 1% by weight of non-specified components.

The term “consisting of” or “consists of” means including the components specified but excluding addition of other components.

Whenever appropriate, depending upon the context, the use of the term “comprises” or “comprising” may also be taken to encompass or include the meaning “consists essentially of” or “consisting essentially of”, and may also be taken to include the meaning “consists of” or “consisting of”.

For the avoidance of doubt, wherein amounts of components in a composition are described in wt %, this means the weight percentage of the specified component in relation to the whole composition referred to. For example, “wherein the oligosaccharide compounds comprise up to 35 wt % of disaccharides” means that 35 wt % of the oligosaccharide compounds in the composition is provided by disaccharides.

The optional features set out herein may be used either individually or in combination with each other where appropriate and particularly in the combinations as set out in the accompanying claims. The optional features for each aspect or exemplary embodiment of the invention as set out herein are also to be read as applicable to any other aspect or exemplary embodiments of the invention, where appropriate. In other words, the skilled person reading this specification should consider the optional features for each exemplary embodiment of the invention as interchangeable and combinable between different exemplary embodiments.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, and drawings), and/or all of the steps of any method or process so disclosed, may be combined in any combination, except combinations where at least some of such features and/or steps are mutually exclusive.

Each feature disclosed in this specification (including any accompanying claims, and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, and drawings), or to any novel one, or any novel combination, of the steps of any method or process so disclosed.

1. A composition comprising oligosaccharide compounds comprising:
(a) at least 8 wt % Gal-(β1-3)-Gal-(β1-4)-X^{a};
(b) at least 3 wt % Gal-(β1-3)-Gal-(β1-3)-X^{b}; and
(c) at least 5 wt % Gal-(β1-3)-Gal-(β1-2)-X^{c};
based on the total weight of oligosaccharide compounds present in the composition;
wherein X^{a}, X^{b} and X^{c} are each independently selected from monosaccharides.

2. The composition according to claim 1, wherein the oligosaccharide compounds further comprise:
(d) at least 3 wt % Gal-(β1-3)-Gal-(β1-3)-Gal-(β1-4)-X^{d};
based on the total weight of oligosaccharide compounds present in the composition;
wherein X^{d}, is selected from monosaccharides.

3. The composition fer-ese-according to claim 1, wherein:
component (a) is present in an amount up to 35 wt %;
component (b) is present in an amount up to 25 wt %; and
component (c) is present in an amount up to 25 wt %;
based on the total weight of oligosaccharide compounds present in the composition.

4. The composition of claim 1, wherein the ratio of the wt % of compound (a) to compound (b) is from 1:1 to 3:1.

5. The composition of claim 1, wherein the ratio of the wt % of compound (a) to compound (c) is from 1:1 to 3:1.

6. The composition of claim 1, wherein the ratio of the wt % of compound (b) to compound (c) is from 2:1 to 1:2.

7. The composition of claim 2, wherein the oligosaccharide compounds further comprise:
(e) at least 5 wt % Gal-(β1-4)-Gal-(β1-4)-X^{e};
based on the total weight of oligosaccharide compounds present in the composition;
wherein X^{e} is selected from monosaccharides.

8. The composition of claim 7, wherein X^{a}, X^{b}, X^{c}, X^{d} and X^{e} are each independently selected from glucose, fucose, arabinose, xylose, rhamnose, mannose, galactose, ribose, lyxose, allose, altrose, gulose, idose, talose, psicose, fructose, sorbose, tagatose, galactosamine, glucosamine and N-Acetylglucosamine or mixtures thereof.

9. The composition of claim 8, wherein X^{a}, X^{b}, X^{c}, X^{d} and X^{e} are each Glc.

10. The composition of claim 8, wherein X^{a}, X^{b}, X^{c}, X^{d} and X^{e} each comprise Fuc.

11. The composition of claim 1, wherein from 40 to 55% of the Gal-Gal linkages in the oligosaccharide compounds are 1-3 linkages.

12. The composition of claim 1 comprising at least 50 wt % of the oligosaccharide compounds.

13. The composition of claim 1, comprising at least 75 wt % of the oligosaccharide compounds.

14. The composition of claim 1, wherein the oligosaccharide compounds comprise at least 25 wt % of trisaccharides.

15. The composition of claim 1, wherein the oligosaccharide compounds comprise at least 10 wt % of tetrasaccharides.

16. The composition of claim 1, comprising 80 wt % of trisaccharides, tetrasaccharides and higher oligosaccharides.

17. The composition of claim 1, comprising 80 wt % of trisaccharides.

18. The composition of claim 1, wherein the composition is in the form of one or more of the following: a pharmaceutical, a nutraceutical, a dietary supplement, a medical food product, a nutritional product, and a food for specific medical purposes.

19-26. (canceled)

27. A method of treating, ameliorating, or preventing a disease or condition associated with elevated levels of ammonia in an individual, the method comprising:
administering to the individual a composition comprising oligosaccharide compounds comprising:
(a) at least 8 wt % Gal-(β1-3)-Gal-(β1-4)-X^{a};
(b) at least 3 wt % Gal-(β1-3)-Gal-(β1-3)-X^{b}; and
(c) at least 5 wt % Gal-(β1-3)-Gal-(β1-2)-X^{c};
based on the total weight of oligosaccharide compounds present in the composition;
wherein X^{a}, X^{b} and X^{c} are each independently selected from monosaccharides.

28. The method of claim 27, wherein the oligosaccharide compounds further comprise:
(d) at least 3 wt % Gal-(β1-3)-Gal-(β1-3)-Gal-(β1-4)-X^{d},
based on the total weight of oligosaccharide compounds present in the composition;
wherein X^{d}, is selected from monosaccharides.
